(19) United States
(12) Patent Application Publication
(10) Pub. No.: US 2003/0181426 A1
(43) Pub. Date: Sep. 25, 2003

(54) Compositions and methods for treating pain using cyclooxygenase-1 inhibitors

(76) Inventor: James C. Eisenach, Winston-Salem, NC (US)

Correspondence Address: MYERS BIGEL SIBLEY & SAJOVEC, PO BOX 37428, RALEIGH, NC, 27627, US

(21) Appl. No.: 10/364,258

(22) Filed: Feb. 11, 2003

Related U.S. Application Data
(60) Provisional application No. 60/356,280, filed on Feb. 11, 2002.

(51) Int. Cl. A61K 31/485; A61K 31/455; A61K 31/445; A61K 31/24; A61K 31/192

(52) U.S. Cl. 514/161; 514/282; 514/317; 514/537; 514/560; 514/355

(57) ABSTRACT

The present invention discloses a method of eliciting an analgesic effect in a subject in need thereof comprising intrathecally administering to the subject a therapeutically effective amount of a cyclooxygenase 1 inhibitor or pharmaceutically acceptable salt thereof in a preservative-free pharmaceutically acceptable carrier. The present invention further discloses pharmaceutical compositions comprising a cylcooxygenase 1 inhibitor or a pharmaceutically acceptable salt thereof and an adjuvant such as an adrenergic agonist, opioid analgesic, local anesthetic, and calcium channel blocker, and combinations thereof in a preservative-free pharmaceutically acceptable carrier. Kits comprising a composition comprising a cyclooxygenase 1 inhibitor or a pharmaceutically acceptable salt thereof in a preservative-free pharmaceutically acceptable carrier in a container suitable for delivery of the composition into an intrathecal administration device are also disclosed herein.

### CROSS REFERENCE TO RELATED APPLICATIONS

This application claims the benefit of U.S. Provisional Application Serial No. 60/356,280 filed Feb. 11, 2002, the disclosure of which is incorporated herein by reference in its entirety.

### STATEMENT OF GOVERNMENT SUPPORT

This invention was made with Government support under grant number NIB GM48085 from the National Institutes of Health. The Government has certain rights to this invention.

### FIELD OF THE INVENTION

The present invention concerns methods, compositions, and kits useful for the treatment of pain in a subject in need thereof.

### BACKGROUND OF THE INVENTION

Since the observation 35 years ago that electrical stimulation induced release of prostaglandins (PGs) from frog spinal cord (Ramwell, 1966), a variety of studies have demonstrated the relevance of spinal PGs in pain. Intrathecal (i.t.) injection of PGs induces thermal and mechanical hyperalgesia (Uda, 1990), blocked by antagonists of PG receptors or by genetic deletion of PG receptor expression (Nakano, 2001). PGs act both pre- (Malmberg, 1994) and post-synaptically (Baba, 2001) to enhance excitatory neurotransmission in the spinal cord, reflecting both exaggerated glutamate, calcitonin gene-related peptide (CGRP), and substance P (sP) release and exaggerated response to their release. This results in reciprocal interactions: i.t. Prostaglandin E2 (PGE2)-induced hypersensitivity is blocked by N-Methyl-D-aspartate (NMDA) receptor antagonists, and i.t. sP- or NMDA-induced hypersensitivity is blocked by cyclooxygenase (COX) inhibitors (Dirig, 1997).

Some noxious stimuli, especially those following acute or chronic inflammation, induce release of spinal PGs, and behavioral responses to these stimuli can be reduced by i.t. injection of COX inhibitors. For example, formalin or zymosan injection in the paw increases PGE2 release in spinal cord microdialysates (Vetter, 2001), and formalin-induced behaviors (Phase II) are reduced by i.t. COX inhibitors (Malmberg, 1992).

The COX-2 isoenzyme is constitutively expressed in spinal cord neurons, and its inhibition results in analgesia. Thus, thermal hyperalgesia from i.t. injection of sP or NMDA as well as that from paw injection of carrageenan is blocked by inhibitors of COX-2, but not COX-1, and these effects occur before any changes in enzyme expression (Yaksh, 2001). Acute inflammation results in increased COX-2 expression in spinal cord (Goppelt-Stiuebe, 1997; Samad, 2001), and there is a predominant, if not exclusive, effect of inhibition of this isoenzyme rather than COX-1 to relieve inflammation-induced hypersensitivity.

Spinally produced PGE2 acts at several receptor subtypes. PG receptor subtype EP1 (EP1) antagonists block allodynia from acute blockade of γ-amino-butyric acid (GABA) receptors with i.t. bicuculline (Zhang, 2001), and from partial sciatic nerve section (Syriatowicz, 1999) or sciatic nerve constriction injury (Kawahara, 2001). Mice lacking the EP1 receptor gene exhibit decreased response to i.p. acetic acid (Stock, 2001) and decreased allodynia from i.t. PGE2 (Nakano, 2001). There is also evidence for other subtypes in pain transmission: PG receptor subtype EP2 (EP2) antagonists selectively block postsynaptic excitation in dorsal horn neurons induced by PGE2 (Baba, 2001), and mice lacking the PG receptor subtype EP3 (EP3) receptor show decreased thermal hyperalgesia from i.t. PGE2 (Nakano, 2001).

Cyclooxygenase (COX) is a known target for non-steroidal anti-inflammatory drugs (NSAIDs) for their anti-inflammatory, anti-pyretic, and analgesic properties (For review, see Insel (1996) in The Pharmacological Basis of Therapeutics New York, pp.617-657). Although there are various mechanisms by which NSAIDs inhibit COX, it is currently appreciated that there are at least two well-characterized forms of cyclooxygenase: cyclooxygenase-1 (COX-1) and cyclooxygenase-2 (COX-2). There are also others such as cyclooxygenase-3 (COX-3) which are being characterized (Chandrasekharan et al., 2002). COX-1 is a constitutive isoform found in blood vessels, stomach and kidney, while COX-2 is induced in the settings of inflammation by cytokines and inflammatory mediators.

In addition to these therapeutic activities, NSAIDs typically possess unwanted side effects, particularly gastrointestinal ulceration and intolerances, blockage; of platelet aggregation, inhibition of uterine motility, inhibition of prostaglandiln-mediated renal function, and hypersensitivity reactions (in Borda I T and Koff R S (eds). NSAIDs: A Profile of Adverse Effects. Philadelphia, Hanley & Belfus, 25-80, 1992.). It has been indicated that the anti-inflammatory action of NSAIDs is due to inhibition of the induced COX-2 enzyme, and that some of the unwanted side effects are due to the inhibition of the constitutive COX-1 enzyme, whose products play a role against damage of the stomach and kidney (Mitchell et al. (1993) Proc. Natl. Acad. Sci. USA 90:11693-11697). Furthermore, studies with the COX-2 selective inhibitor, NS-398, compared to indomethancin indicate that selective COX-2 inhibition is anti-inflammatory but not ulcerogenic in a rat animal model (Mansferrer et al. (1994) Proc. Natl. Acad. Sci U.S.A. 91:3228-3232). Thus, many of the current efforts in the design of NSAIDs for analgesia and anti-inflammatory effects have aimed at more selective inhibitors of COX-2 versus COX-1, due to the apparent lower incidence of ulcerogenic side-effects of COX-2 selective inhibitors.

NSAIDs in use today have varying abilities to inhibit COX-1 and COX-2, respectively. As known in the art, NSAIDs vary from those that preferentially inhibit COX-2 (e.g. nimesulide and 6-methoxy-2-napthyl acetic acid), those that show no or small preferences in inhibition of COX-1 and COX-2 (e.g., ibuprofen), to those that preferentially inhibit COX-1 (e.g., flubiprofen and indomethancin). Although the role of COX-1 inhibition has been implicated in the side effects of NSAIDs, it has not been implicated to any significant extent to play a role in analgesia. However, Mazario et al. ((2001) Neuropharmacology 40:937-946) have shown that COX-2 selective inhibitors, celecoxib and rofecoxib, have no effect in reducing nociceptive responses both in normal and monoarthritic rats, and in mice with paw inflammation. Similarly, in studies by Tegeder et al. ((2001) J. Neurochem. 79:777-786), the COX-2 selective inhibitor, celecoxib, exhibited no significant effect on formalin-evoked nociceptive behavior and spinal PGE(2) release in the induction of hyperalgesia and allodynia. Finally, Ochi et al. ((2000) Eur. J. Pharmacol. 391:49-54) examined the pharmacological profile of the COX-1 specific inhibitor, FR122047, in chemical nociceptive models, and saw a dose-dependent analgesic effect against the acetic acid-induced writhing response in mice, whereas the COX-2 specific inhibitor, NS-398, had no effect in the examined COX-1 sensitive pain models.

The current understanding, or ‘dogma’ of how spinal prostaglandins mediate the perception of pain is schematically illustrated in FIG. 1.

In view of the foregoing, there remains a need for new compositions and methods of eliciting analgesia.

### SUMMARY OF THE INVENTION

In general, the present invention provides a method of eliciting an analgesic effect in a subject in need thereof, comprising intrathecally administering to the subject a therapeutically effective amount of a cyclooxygenase 1 inhibitor with or without an adjuvant of this invention in a preservative-free pharmaceutically acceptable carrier.

A further aspect of the present invention is a method of eliciting an analgesic effect in a subject in need thereof, comprising intrathecally administering an effective amount of ketorolac to the subject in a preservative-free pharmaceutically acceptable carrier.

Another aspect of the present invention provides a pharmaceutical composition comprising a cyclooxygenase 1 inhibitor and an adjuvant which can be, for example, an adrenergic agonist, an opioid analgesic, a local anesthetic, and a calcium channel blocker, and/or combinations thereof in a preservative-free pharmaceutically acceptable carrier.

A still further aspect of the present invention provides a pharmaceutical composition comprising ketorolac and an adjuvant which can be, for example, an adrenergic agonist, an opioid analgesic, a local anesthetic, and a calcium channel blocker, and/or combinations thereof in a preservative-free pharmaceutically acceptable carrier.

A further aspect of the present invention provides a pharmaceutical composition comprising ketorolac and clonidine in a preservative-free pharmaceutically acceptable carrier.

Another aspect of the present invention provides a pharmaceutical composition comprising ketorolac and fentanyl in a preservative-free pharmaceutically acceptable carrier.

A still further aspect of the present invention provides a pharmaceutical composition comprising ketorolac and lidocaine in a preservative-free pharmaceutically acceptable carrier.

A further aspect of the present invention provides a kit comprising a composition comprising a cyclooxygenase 1 inhibitor in a preservative-free pharmaceutically acceptable carrier in a container suitable for delivery of the composition into an intrathecal administration device.

Another aspect of the present invention provides a kit comprising a composition comprising ketorolac in a preservative-free pharmaceutically acceptable carrier in a container suitable for delivery of the composition into an intrathecal administration pump.

A still further aspect of the present invention is the use of the compositions as described above for the preparation of a medicament for eliciting analgesia as described above.

The foregoing and other aspects of the present invention are explained in greater detail in the specification set forth below.

### BRIEF DESCRIPTION OF THE DRAWINGS

FIG. 1: The current dogma on spinal PGs: (1) C and Aδ fibers release sP and glutamate, which act on NK1 and NMDA receptors to increase free Ca²⁺ (2) activates cPLA₂ to release arachidonic acid (AA), a substrate for constitutive (3) COX2, resulting in PG synthesis. PGE2 (4) enhances presynaptic release and (5) depolarizes postsynaptic membrane.

FIG. 2: New hypotheses for spinal PGs: (1) Afferent activity after surgery induces glutamate release, which stimulates AMPA receptors on postsynaptic cells and glia (2) AMPA activation in glia stimulates iPLA₂, releasing arachidonic acid acted on by COX1 (3). COX1 activity results in PGE2 synthesis, sensitizing pre- and postsynaptic elements (4). IL-1β, from peripheral sites and glia, sensitizes processing and induces COX.

FIG. 3: Effect of intrathecal ketorolac on volunteers' pain magnitude report to thermal stimuli (high intensity in circles, low intensity in squares).

FIG. 4: I.t. ketorolac in Brennan model. Withdrawal threshold to von Frey filament testing before surgery (Pre-Surg) and after surgery for 3 postoperative days (POD)

FIG. 5: Post-laparotomy activity. Ambulatory and vertical spontaneous activity are reduced 24 hr after laparotomy compared to sham in saline treated animals (left panel). The reduction in ambulatory, but not vertical counts is blocked by IV morphine, 3 mg/kg (left panel). Laparotomy decreases the number of sucrose pellets self-administered in a 1 hr period for 2 days after surgery (middle panel), and prolongs the time to self-administration of pellets in each trial for up to 10 days after surgery (right panel).

FIG. 6: Effect of intrathecal ketorolac on behavior after laparotomy. Compared to saline, ketorolac increased both ambulatory and vertical activity.

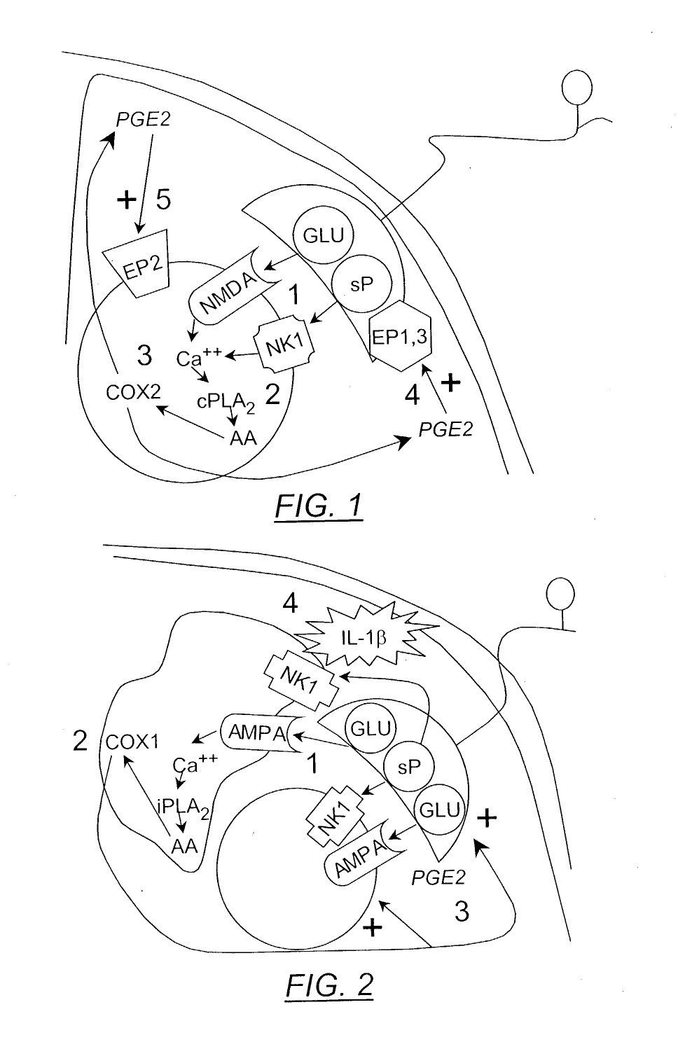

### DETAILED DESCRIPTION OF PREFERRED EMBODIMENTS

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention pertains. The terminology used in the description of the invention herein is for the purpose of describing particular embodiments only and is not intended to be limiting of the invention. As used in the description of the invention and the claims set forth herein, the singular forms “a,” “an,” and “the” are intended to include the plural forms as well, unless the context clearly indicates otherwise.

All publications, patent applications, patents and other references cited herein are incorporated by reference in their entireties for the teachings relevant to the sentence and/or paragraph in which the reference is presented.

As used herein, the phrase “eliciting an analgesic effect” refers to any type of action or treatment that imparts a pain-relieving effect upon a subject afflicted with or experiencing the sensation of pain or at risk of experiencing pain or the sensation of pain, including reducing the sensation of pain or the report of pain or delaying the development of the sensation of pain or the report of pain. That pain is relieved (e.g., by complete or partial abolition of pain symptoms) by administering the compositions of this invention according to the methods provided herein can be determined by art-known assays designed to measure, either quantitatively or qualitatively, the sensation of pain or the report or perception of pain. The sensation of pain or the report of pain can be evaluated by protocols understood by those of ordinary skill in the art to which this invention pertains (See for example, Stubhaug A, 1997 and Silverman D G et al. 1993). For example, pain can be quantitatively assessed using a visual analog scale (VAS) which comprises a 10 cm line with “No Pain” above one end and “Worst Pain Imaginable” on the other end. Alternatively, a mechanical VAS device a (slide-rule type device) can be used to assess pain. Pain after surgery can be assessed using either the line or mechanical VAS. The phrase “eliciting an analgesic effect” further includes prophylactic treatment of the subject to prevent the onset of the sensation of pain or the report of pain. As used herein, “eliciting an analgesic effect” can include a complete and/or partial abolition of the sensation of pain or the report of pain. For example, an analgesic effect can include any reduction in the sensation and/or symptoms of pain including reducing the intensity and/or unpleasantness of the perceived pain.

As used herein, “pain” refers to all types of pain and the methods and compositions of this invention are directed to eliciting an analgesic effect in a subject to treat a specific type of pain or more than one type of pain as described herein. Pain can be acute or chronic pain. Pain as described herein can include sensations such as discomfort, sensitivity, burning, pinching, stinging, etc. Examples of types of pain that can be treated according to the present invention include, but are not limited to, inflammation, visceral pain, neuropathic pain, lower back pain, incisional pain (pain due to or caused by an incision), post-surgical pain, and post-surgical incisional pain. Moreover, the term “pain” also refers to nociceptive pain or nociception.

“Therapeutically effective amount” as used herein refers to an amount of a compound or composition that is sufficient to produce the desired therapeutic effect. The therapeutically effective amount will vary with the age and physical condition of the subject, the severity of the disorder, the duration of the treatment, the nature of any concurrent treatment, the pharmaceutically acceptable carrier used, and like factors within the knowledge and expertise of those skilled in the art. An appropriate “therapeutically effective amount” in any individual case can be determined by one of ordinary skill in the art by reference to the pertinent texts and literature and/or by using routine experimentation. (See, for example, Remington, The Science And Practice of Pharmacy (9^{th} Ed. 1995).

According to the present invention, methods of this invention comprise administering an effective amount of a composition of the present invention as described above to the subject. The effective amount of the composition, the use of which is in the scope of present invention, will vary somewhat from subject to subject, and will depend upon factors such as the age and condition of the subject and the route of delivery. Such dosages can be determined in accordance with routine pharmacological procedures known to those skilled in the art. For example, COX1 inhibitors and/or adjuvants of the present invention can be administered to the subject in an amount ranging from a lower limit of about 0.01, 0.02, 0.03, 0.04, 0.05, 0.06, 0.07, 0.08, 0.09, or 0.1 mg to an upper limit of about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mg in a single dose; in an amount ranging from a lower limit of about 0.01, 0.02, 0.03, 0.04, 0.05, 0.06, 0.07, 0.08, 0.09, or 0.1 mg to an upper limit of about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0 or 20.0 mg in a 24 hour period; and as much as 30, 40, 50, 60, 70, 80, 90, 100, 200, 300, 400, 500 mg or more over a prolonged period of time with a medical infusion pump or similar device designed for delivery of a substance over a prolonged period. The frequency of administration can be one, two, three, four, five times or more per day or as necessary to control the condition. The duration of therapy depends on the type of condition being treated and can be for as long as the life of the subject.

“Cyclooxygenase 1 inhibitor” as used herein refers to an anti-inflammatory agent that inhibits prostaglandin biosynthesis. More specifically, COX-1 inhibitors are those that exhibit a lower IC₅₀ for the COX-1 isozyme than for other COX isozymes. Thus, the agent can be completely selective for COX-1, or can only be relatively selective for COX-1 in comparison to its selectivity for other COX isozymes. Examples of COX-1 inhibitors of this invention can include, but are not limited to, ketorolac, piroxicam, diclofenac, naproxen, meclofenamate, indomethancin, phenylbutazone, flubiprofen, experimental COX-1 inhibitor NS398, the COX-1 selective inhibitors SC-560, SC-58560, and FR122047, and any other COX 1 inhibitors now known or later identified.

As used herein, a “pharmaceutically acceptable carrier” according to the present invention is a component such as a carrier, diluent, or excipient of a composition that is compatible with the other ingredients of the composition in that it can be combined with the compounds and/or compositions of the present invention without eliminating the biological activity of the compounds or the compositions, and is suitable for use in subjects as provided herein without undue adverse side effects (such as toxicity, irritation, allergic response, and death). Side effects are “undue” when their risk outweighs the benefit provided by the pharmaceutical composition. Non-limiting examples of pharmaceutically acceptable components include, without limitation, any of the standard pharmaceutical carriers such as phosphate buffered saline solutions, water, emulsions such as oil/water emulsions or water/oil emulsions, microemulsions, and various types of wetting agents.

As used herein, “preservative-free” refers to the substantial absence of chemical, antibacterial, antimicrobial, or antioxidative additives, or the like from the pharmaceutically acceptable carriers of the present invention. “Substantial absence” can mean that no preservative is present in the compositions or that trace amounts can be present that impart no detectable effect otherwise attributable to a preservative.

“Adjuvant” as used herein refers to a compound that, when used in combination with the compounds and/or compositions of the present invention, preferably augments or otherwise alters or modifies the resultant pharmacological and/or physiological responses.

“Kit” as used herein refers to an assembly of components. The assembly of components can be a partial or complete assembly.

As used herein, “administered with” means that the composition of the present invention and at least one other adjuvant are administered at times sufficiently close that the results observed are indistinguishable from those achieved when the compounds are administered at the same point in time. The compounds can be administered simultaneously (i.e., concurrently) or sequentially. Simultaneous administration can be carried out by mixing the compounds prior to administration, or by administering the compounds at the same point in time. Such administration can be at different anatomic sites or using different routes of administration. The phrases “concurrent administration,” “administration in combination,” “simultaneous administration” or “administered simultaneously” can also be used interchangeably and mean that the compounds are administered at the same point in time or immediately following one another. In the latter case, the two compounds are administered at times sufficiently close that the results produced are synergistic and/or are indistinguishable from those achieved when the compounds are administered at the same point in time. Alternatively, a COX-1 inhibitor of this invention can be administered separately from the administration of an adjuvant of this invention, which can result in a synergistic effect or a separate effect.

In view of the foregoing, embodiments according to the present invention relate to a method of eliciting an analgesic effect in a subject in need thereof, comprising intrathecally administering to the subject a therapeutically effective amount of a COX-1 inhibitor in a preservative-free pharmaceutically acceptable carrier. Examples of COX-1 inhibitors include, but are not limited to, ketorolac, piroxicam, diclofenac, naproxen, meclofenamate, indomethancin, phenylbutazone, flubiprofen, experimental COX-1 inhibitor NS398, the COX-1 selective inhibitors SC-560, SC-58560, and FR122047, and any other COX-1 inhibitors now known or later identified. COX-1 inhibitors of the present invention can be administered as a single COX-1 inhibitor or as a combination of COX-1 inhibitors comprising the COX-1 inhibitors as described herein. In some embodiments, the COX-1 inhibitor can be ketorolac, piroxicam, and/or diclofenac. In certain embodiments, the COX-1 inhibitor can be ketorolac.

In some embodiments, the COX-1 inhibitor is administered with an adjuvant. The COX-1 inhibitor can be administered with a single adjuvant or a combination of adjuvants. Furthermore, a combination of COX-1 inhibitors can be administered with a single adjuvant or a combination of adjuvants. Examples of adjuvants include, but are not limited to, adrenergic agonists, opioid analgesics, local anesthetics, calcium channel blockers, and combinations thereof. Representative non-limiting examples of adrenergic agonists include α₂-agonists such as clonidine, apraclonidine, tizanidine, guanfacine, guanabenz, and methyldopa.

Representative non-limiting examples of opioid analgesics include alfentanil, buprenorphine, butorphanol, codeine, dezocine, dihydrocodeine, fentanyl, hydrocodone, hydromorphone, levorphanol, meperidine (pethidine), methadone, morphine, nalbuphine, oxycodone, oxymorphone, pentazocine, bremazocine, propiram, propoxyphene, sufentanil, tramadol, endorphins, enkephalins, deltorphins, dynorphins and analogs and derivatives thereof, and other naturally occurring and synthetic agonists also possessing an affinity for opioid receptors as understood by those of ordinary skill in the art to which the present invention pertains.

Representative non-limiting examples of local anesthetics include lidocaine, prilocaine, bupivacaine, mepivacaine, ropivacaine and related local anesthetic compounds having various substituents on the ring system or amine nitrogen; the aminoalkyl benzoate compounds, such as procaine, chloroprocaine, propoxycaine, hexylcaine, tetracaine, cyclomethycaine, benoxinate, butacaine, proparacaine, and related local anesthetic compounds; cocaine and related local anesthetic compounds; amino carbonate compounds such as diperodon and related local anesthetic compounds; N-phenylamidine compounds such as phenacaine and related anesthetic compounds; N-aminoalkyl amid compounds such as dibucaine and related local anesthetic compounds; aminoketone compounds such as falicaine, dyclonine and related local anesthetic compounds; and amino ether compounds such as pramnoxine, dimethisoquien, and related local anesthetic compounds as understood by those of ordinary skill in the art to which the present invention pertains.

Examples of calcium channel blockers according to the present invention include, but are not limited to, compounds effective to interfere with the flow of calcium ions down the electrochemical gradient of one or more calcium channels. For example, N-type calcium channels are unique to neurons, and are characterized by single channel conductance and sensitivity to ω-conotoxin. (Bean, Ann. Rev. Physiol. 51:367-384 (1989)). Potent and selective N-channel blocking compounds currently known are the conopeptides which are peptide toxins produced by pisciverous marine snails of the genus Conus. U.S. Pat. No. 5,051,403, incorporated herein by reference, describes how to make and use certain ω-conopeptides having defined binding/inhibitory properties, and specifically, the synthetic ω-conotoxin peptide MVIIA (SNX-111) (ziconotide) and derivatives thereof (e.g., SNX-194). Such calcium channel blockers now known and later identified represent non-limiting examples of calcium channel blockers of the present invention. Moreover, the term “antagonist” is synonymous with the term “blocker” in this context.

In certain embodiments of the present invention, the pharmaceutically acceptable carrier is preservative free. For example, the pharmaceutically acceptable carrier can be characterized by the substantial absence of chemical, antibacterial, antimicrobial, or antioxidative additives or the like (e.g., contain less than about 5.0, 4.0, 3.0, 2.0, 1.0, 0.5, 0.1, 0.05, 0.01, or even 0.00 percent by weight of a preservative). Further, such formulations are substantially or essentially free of alcohols such as ethanol (e.g., contain less than about 5.0, 4.0, 3.0, 2.0, 1.0, 0.5, 0.1, 0.05, 0.01, or even 0.00 percent by weight of alcohols such as ethanol). Examples of suitable formulations include, but are not limited to, formulations comprising, consisting of or consisting essentially of the active agent and physiological saline solution (optionally including other typical ingredients such as other active agents and buffers).

In some embodiments of the present invention, the COX-1 inhibitors of the present invention can be administered with adjuvants such as antidepressants, sedatives, and hypnotics. Such adjuvants can be administered to render calming, sedation, sleep, antiepileptic agents, unconsciousness, surgical anesthesia, and coma to patients wherein Such an additional effect is desired.

The invention also relates to pharmaceutical compositions comprising a COX-1 inhibitor and an adjuvant such as an adrenergic agonist, opioid analgesic, local anesthetic, and calcium channel blocker, and combinations thereof in a preservative-free pharmaceutically acceptable carrier. Representative non-limiting COX-1 inhibitors are previously described herein. In certain embodiments, the COX-1 inhibitor can be ketorolac, piroxicam, or diclofenac. In some embodiments, the COX-1 inhibitor can be a combination of COX-1 inhibitors. In other embodiments, the COX-1 inhibitor is ketorolac. Representative non-limiting examples of adrenergic agonists, opioid analgesics, local anesthetics, and calcium channel blockers are previously described herein. In certain embodiments, the adjuvants are clonidine, fentanyl, lidocaine, or combinations thereof. As discussed herein, the pharmaceutically acceptable carrier can be characterized by the substantial absence of chemical, antibacterial, antimicrobial, or antioxidative additives, or the like.

Thus, in further embodiments, the present invention provides a method of eliciting an analgesic effect in a subject in need thereof, comprising intrathecally administering to the subject a therapeutically effective amount of a cyclooxygenase 1 inhibitor or pharmaceutically acceptable salt thereof and an adjuvant such as an adrenergic agonist, opioid analgesic, local anesthetic, and calcium channel blocker, and combinations thereof in a pharmaceutically acceptable carrier, which in certain embodiments can be preservative-free or can contain a preservative. Representative non-limiting COX-1 inhibitors are previously described herein. In certain embodiments, the COX-1 inhibitor can be ketorolac, piroxicam, or diclofenac. In some embodiments, the COX-1 inhibitor can be a combination of COX-1 inhibitors. In other embodiments, the COX-1 inhibitor is ketorolac. Representative non-limiting examples of adrenergic agonists, opioid analgesics, local anesthetics, and calcium channel blockers are previously described herein. In certain embodiments, the adjuvants are clonidine, fentanyl, lidocaine, or combinations thereof.

In still other embodiments, the present invention provides a kit comprising a composition comprising a COX-1 inhibitor in a pharmaceutically acceptable carrier that can be preservative free or can include a preservative, in a container suitable for delivery of the composition into an intrathecal administration device. Representative non-limiting COX-1 inhibitors are previously described herein. In certain embodiments, the COX-1 inhibitor can be ketorolac, piroxicam, or diclofenac. In some embodiments, the COX-1 inhibitor can be a combination of COX-1 inhibitors. In other embodiments, the COX-1 inhibitor is ketorolac. As discussed herein, the pharmaceutically acceptable carrier can be characterized by the substantial absence of chemical, antibacterial, antimicrobial, or antioxidative additives, or the like. Moreover, the kits can comprise one or more COX-1 inhibitors as described herein and one or more adjuvants as described herein, in any combination.

The intrathecal administration device according to the present invention can be any mechanism enabling the intrathecal administration of the composition to the subject as known to those skilled in the art. Examples of intrathecal administration devices include, but are not limited to, pumps (implantable or external devices), epidural injectors, spinal tap injection syringes or injection apparatus, or an intrathecal administration/injection apparatus (e.g., a catheter and/or a reservoir operably associated with the catheter), etc. In certain embodiments, the intrathecal administration device is a pump, syringe, catheter, or a reservoir operably associated with a connecting device such as a catheter, tubing, or the like. Containers suitable for delivery of the composition into the intrathecal administration device pertain to instruments of containment which can be used to deliver, place, attach, or insert the composition into the intrathecal device for intrathecal delivery of the composition to the subject. Such containers include, but are not limited to, vials, ampules, tubes, capsules, bottles, syringes, and bags.

In some embodiments, the kit further comprises an adjuvant such as an adrenergic agonist, opioid analgesic, local anesthetic, calcium channel blocker, and/or combinations thereof. In other embodiments, kits according to the present invention can comprise the COX-1 inhibitor and the adjuvant in separate containers or in the same container. In certain embodiments, the adjuvants are clonidine, fentanyl, lidocaine, or combinations thereof. Moreover, kits according to the present invention can partially or completely contain components for intrathecal administration of the compositions of the present invention as described herein. Kits can further include accessory items such as tubing, stoppers and the like. As non-limiting examples, a kit can comprise a COX-1 inhibitor in a vial, a kit can comprise a COX-1 inhibitor in a vial along with a syringe, a kit can comprise a COX-1 inhibitor in a vial along with a syringe, a delivery bag, catheter, and suitable tubing, or a kit can comprise a COX-1 inhibitor in an ampule and an adjuvant in a vial along with a medical infusion pump. Thus, a kit can contain some or all of the components required to intrathecally administer the compositions of the present invention to a subject.

The active compounds disclosed herein can be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example, hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine; and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

The active compounds described above can be formulated for administration in accordance with known pharmacy techniques. See, e.g., Remington, The Science And Practice of Pharmacy (9^{th} Ed. 1995). In the manufacture of a pharmaceutical composition according to the present invention, the active compound (including the physiologically acceptable salts thereof is typically admixed with, inter alia, an acceptable carrier. The carrier must, of course, be acceptable in the sense of being compatible with any other ingredients in the formulation and must not be deleterious to the patient. The carrier can be a solid or a liquid, or both, and is preferably formulated with the compound as a unit-dose formulation, for example, a tablet, which can contain from 0.01% or 0.5% to 95% or 99%, or any value between 0.01% and 99%, by weight of the active compound. One or more active compounds can be incorporated in the compositions of the invention, which can be prepared by any of the well-known techniques of pharmacy, comprising admixing the components, optionally including one or more accessory ingredients. Moreover, the carrier can be preservative free, as described herein above.

In some embodiments, the COX-1 inhibitor provided by the present invention comprises a lower limit ranging from about 0.01, 0.02, 0.03, 0.04, 0.05, 0.06, 0.07, 0.08, 0.09, 1.0, 2.0, 3.0, 4.0, 5.0, 6.0, 7.0, 8.0, 9.0, and 10% to an upper limit ranging from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100% by weight of the composition. In some embodiments, the COX-1 inhibitor comprises from about 0.05% to about 100% by weight of the composition.

The formulations of the present invention can include those suitable for oral, rectal, topical, buccal (e.g., sub-lingual), vaginal, parenteral (e.g., subcutaneous, intramuscular, intradermal, intravenous, or intrathecal), topical (i.e., both skin and mucosal surfaces, including airway surfaces) and transdermal administration, although the most suitable route in any given case will depend on the nature and severity of the condition being treated and on the nature of the particular active compound which is being used.

Preferred routes of parenteral administration include intrathecal injection and intraventricular injection into a ventricle of the brain.

Formulations suitable for oral administration can be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations can be prepared by any suitable method of pharmacy which includes bringing into association the active compound and a suitable carrier (which can contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet can be prepared by compressing or molding a powder or granules containing the active compound, optionally with one or more accessory ingredients. Compressed tablets can be prepared by compressing, in a suitable machine, the compound in a free-flowing form, such as a powder or granules optionally mixed with a binder, lubricant, inert diluent, and/or surface active/dispersing agent(s). Molded tablets can be made by molding, in a suitable machine, the powdered compound moistened with an inert liquid binder.

Formulations of the present invention suitable for parenteral administration comprise sterile aqueous and non-aqueous injection solutions of the active compound, which preparations are preferably isotonic with the blood of the intended recipient. These preparations can contain, buffers and solutes which render the formulation isotonic with the blood of the intended recipient. Aqueous and non-aqueous sterile suspensions can include suspending agents and thickening agents. The formulations can be presented in unit\dose or multi-dose containers, for example sealed ampoules and vials, and can be stored in a freeze-dried (lyophilized) condition requiring only the addition of the sterile liquid carrier, for example, saline or water-for-injection immediately prior to use. Extemporaneous injection solutions and suspensions can be prepared from sterile powders, granules and tablets of the kind previously described.

For example, in one aspect of the present invention, there is provided an injectable, stable, sterile composition comprising active compounds, or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable can be employed in sufficient quantity to emulsify the compound or salt in an aqueous carrier. Non-limiting examples of agents that contribute to the pharmaceutical acceptability of the compositions of the present invention include normal saline, phosphatidyl choline, and glucose. In some embodiments, the pharmaceutically acceptable carrier can be normal saline. In other embodiments, the pharmaceutically acceptable carrier can be normal saline with up to 0.0, 0.01, 0.02, 0.03, 0.04, 0.05, 0.06, 0.07, 0.08, 0.09, 1.0, 2.0, 3.0, 4.0, 5.0, 6.0, 7.0, 8.0, 9.0, 10, 11, 12, 13, 14, 15, 16, 17, 18, 19, and 20%, and any value between 0.01% and 20%, glucose.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Pharmaceutical Research 3(6):318 (1986)) and typically take the form of an optionally buffered aqueous solution of the active compound. Suitable formulations comprise citrate or bis\tris buffer (pH 6) or ethanol/water and contain from 0.1 to 0.2M active ingredient.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same can be incorporated into lipid vesicles. In such an instance, due to the water solubility of the compound or salt, the compound or salt will be substantially entrained within the hydrophilic center or core of the liposomes. The lipid layer employed can be of any conventional composition and can either contain cholesterol or can be cholesterol-free. When the compound or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations containing the compounds disclosed herein or salts thereof, can be lyophilized to produce a lyophilizate which can be reconstituted with a pharmaceutically acceptable carrier, such as water, to regenerate a liposomal suspension.

Other pharmaceutical compositions can be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to active agents or their salts, the pharmaceutical compositions can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions can contain microbial preservatives. Useful microbial preservatives include methylparaben, propylparaben, and benzyl alcohol. The microbial preservative is typically employed when the formulation is placed in a vial designed for multidose use. The pharmaceutical compositions of the present invention can be lyophilized using techniques well known in the art.

Preferred routes of administration of the present invention are by injection into the cerebrospinal (CSF) fluid of the subject, such as by intrathecal injection or epidural injection and intraventricular injection into a ventricle of the brain. Injection into the cerebrospinal fluid can be carried out in accordance with known techniques, including but not limited to those described in U.S. Pat. No. 6,333,037, the disclosure of which is incorporated herein by reference in its entirety.

Therapeutic administration of certain drugs intraspinally, that is to either the epidural space or to the intrathecal space, is known. Administration of a drug directly to the intrathecal space can be, for example, by spinal tap injection or by catheterization. Intrathecal drug administration can avoid the inactivation of some drugs when taken orally as well as and the systemic effects of oral or intravenous administration. Additionally, intrathecal administration permits use of an effective dose which is only a fraction of the effective dose required by oral or parenteral administration. Furthermore, the intrathecal space is generally wide enough to accommodate a small catheter, thereby enabling chronic drug delivery systems. Thus, for example, it is known to treat spasticity by intrathecal administration of baclofen. Additionally, it is known to combine intrathecal administration of baclofen with intramuscular injections of botulinum toxin for the adjunct effect of intramuscular botulinum for reduced muscle spasticity. Furthermore, as another example, it is known to treat pain by intraspinal administration of the opioids morphine and fentanyl, as set forth in Gianno et al. (Intrathecal Drug Therapy for Spasticity and Pain, Springer-Verlag (1996)), the contents of which publication are incorporated herein by reference in their entirety.

The current method for intrathecal treatment of chronic pain is by use of an intrathecal pump, such as the SynchroMed® Infusion System, a programmable, implanted pump available from Medtronic, Inc., of Minneapolis, Minn. A pump is required because the antinociceptive or antispasmodic drugs in current use have a short duration of activity and must therefore be frequently readministered, which readministration is not practically carried out by daily spinal tap injections. The pump is surgically placed under the skin of the patient's abdomen. One end of a catheter is connected to the pump, and the other end of the catheter is threaded into a CSF-filled subarachnoid or intrathecal space in the patient's spinal cord. The implanted pump can be programmed for continuous or intermittent infusion of the drug through the intrathecally located catheter. Complications can arise due to the required surgical implantation procedure and the known intrathecally administered drugs for pain have the disadvantages of short duration of activity, lipid solubility which permits passage out of the intrathecal space and systemic transport and/or diffusion to higher central nervous system (CNS) areas with potential respiratory depression resulting.

The intraspinal administration of the active agent is preferably by intrathecal administration, such as intrathecally to a cranial, cervical, thoracic, lumbar, sacral or coccygeal region of the central nervous system and administration can include accessing a subarachnoid space of the central nervous system of the mammal, and injecting the active agent into the subarachnoid space. The accessing step can be carried out by spinal tap.

Alternately, intraspinal administration can include catheterization of a subarachnoid space of the central nervous system of the mammal, followed by injection of the active agent through a catheter inserted by the catheterization step into the subarachnoid space. Note that prior to the injecting step there can be the step of attaching to or implanting in the mammal an administration means for administering the active agent to the central nervous system of the mammal. The administration means can be made up of a reservoir of the active agent, where the reservoir is operably connected to a pump means for pumping an aliquot of the active agent out of the reservoir and into an end of the catheter in the subarachnoid space.

Subjects suitable to be treated according to the present invention include, but are not limited to, avian and mammalian subjects, and are preferably mammalian. Mammals of the present invention include, but are not limited to, canines, felines, bovines, caprines, equines, ovines, porcines, rodents (e.g. rats and mice), lagomorphs, primates, humans, and the like, and mammals in utero. Any mammalian subject in need of being treated according to the present invention is suitable. Human subjects are preferred. Human subjects of both genders and at any stage of development (i.e., neonate, infant, juvenile, adolescent, adult) can be treated according to the present invention.

Illustrative avians according to the present invention include chickens, ducks, turkeys, geese, quail, pheasant, ratites (e.g., ostrich) and domesticated birds (e.g., parrots and canaries), and birds in ovo.

Moreover, suitable subjects of this invention include those that have not previously been afflicted with pain and/or the sensation of pain, those that have previously been determined to be at risk of experiencing pain and/or the sensation of pain, and those that have been initially diagnosed or identified as being afflicted with or experiencing pain and/or the sensation of pain.

The present invention is primarily concerned with the treatment of human subjects, but the invention can also be carried out on animal subjects, particularly mammalian subjects such as mice, rats, dogs, cats, livestock and horses for veterinary purposes, and for drug screening and drug development purposes. Suitable subjects include subjects undergoing surgery, for which the COX-1 inhibitor of this invention is administered with or without an adjuvant and/or in combination with a local anesthetic to produce spinal anesthesia.

The present invention will now be described with reference to the following examples. It should be appreciated that these examples are for the purposes of illustrating aspects of the present invention, and do not limit the scope of the invention as defined by the claims.

### EXAMPLE 1

The present invention is schematically illustrated in FIG. 2. Several aspects regarding spinal PGs after inflammation are under general question, and others are unique to the present study examining postoperative sensitivity. Unlike the exclusive role of COX-2 in inflammation-induced spinal sensitization, COX-2 selective antagonists have minimal or no effects i.t. after surgery (Yamamoto, 1999). Preliminary data demonstrated activity of the COX-1 preferring inhibitor ketorolac in two models of postoperative hypersensitivity. Unlike mechanical hypersensitivity from inflammation following surgery which is insensitive to i.t. NMDA antagonists, but is sensitive to AMPA/kainate antagonists (Zahn, 1998), mechanical hypersensitivity following spinal AMPA injection, perhaps mimicking postoperative hypersensitivity, is blocked by i.t. COX inhibitors, although subtype specific agents have not been examined (Meller, 1996).

It is proposed that a major mechanism of action of i.t. ketorolac after surgery reflects inhibition of COX-1 activity in glia. Increased COX-1 expression after surgery is localized in dorsal horn cells with glial morphology. Spinal cord glial activation has recently been demonstrated following nerve injury (Colburn, 1997; Sweitzer, 2001) and tissue injury/inflammation (Fu, 2000). Signals for glial activation include excitatory neurotransmitters released from nociceptive afferents as well as cytokines and growth factors, transported from the site of peripheral injury and released centrally (Okamoto, 2001). Glia enhance glutamate release and also release a variety of substances which activate and sensitize spinal cord neurons, including cytokines, growth factors, nitric oxide, and prostaglandins, (Watkins, 2001). Although glial activation and its role in COX expression has been studied in various inflammatory and injury models (Watkins, 2001), it has not previously been examined after surgery.

Although often stated as being nonselective, ketorolac is actually one of the most selective COX-1 antagonists of all currently available agents (Warner, 1999), and pain relief after tooth extraction surgery in humans correlates better with COX-1 than COX-2 inhibition (McCormack, 1994). COX-1, but not COX-2 knockout mice have reduced nociceptive responses to i.p. acetic acid (Ballou, 2000). Cytokines and growth factors, known to be released by activated glia, are capable of upregulating COX-1 expression (Versteeg, 1999). iPLA₂, the isoenzyme important in phospholipid remodeling, has been recognized to respond to increases in intracellular Ca²⁺ in vivo, and its activation results in PGE2 synthesis by COX-1, but not by COX-2 (Murakami, 1999). Glial activation results in rapid changes in cell morphology and sprouting of multiple processes, likely associated with increased iPLA2 activity, and perhaps explaining the large increase in COX-1 immunostaining in these cells after surgery observed herein.

It is not disputed that peripheral inflammation produces spinal sensitization by a COX-2 dependent process; that NMDA-induced hyperalgesia is blocked by COX-2 inhibitors; or that humans obtain pain relief after surgery from systemic administration of COX-2 selective inhibitors. The data provided is strong evidence that spinal COX-1 expression is elevated after surgery, and that spinal COX-1 can be an important target for postoperative pain treatment, particularly by i.t. ketorolac.

Ketorolac Data

A. Human Experience

A preclinical toxicity screening was completed for i.t. ketorolac in rats and dogs. Although there was no evidence for spinal neurotoxicity, doses were large enough in some animals to produce gastrointestinal ulceration and bleeding. Thus, dose level was limited by systemic actions rather than any local toxicity. I.t. ketorolac reduced CSF PGE2 concentrations in conscious dogs by >90% within 30 min of injection.

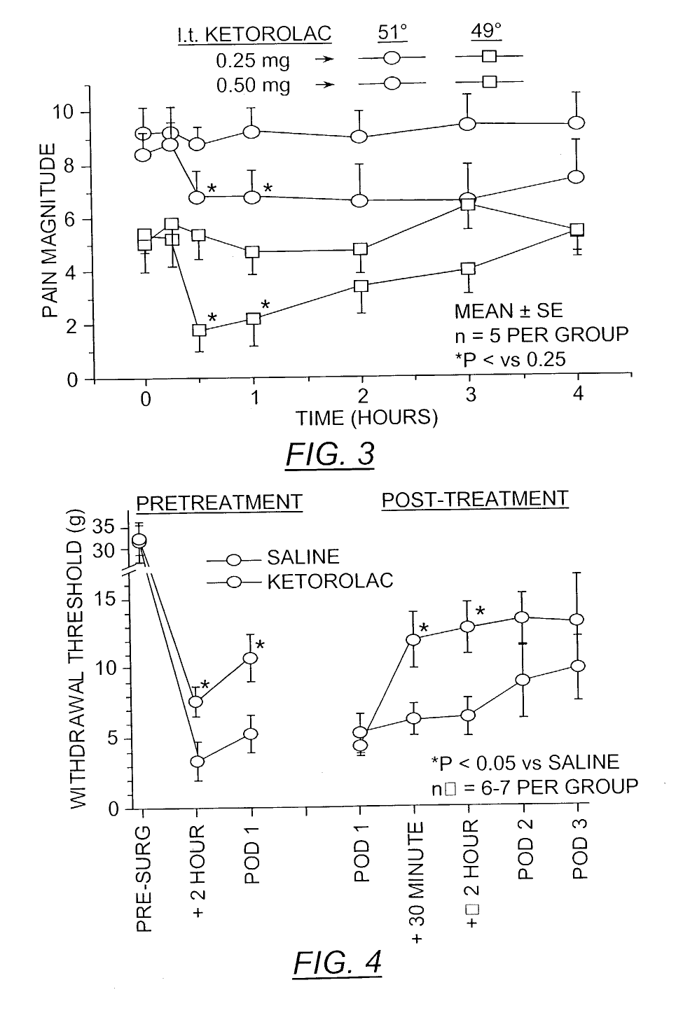

Based on these data, an IND was approved by the FDA in May 2001. A Phase I safety trial has been completed. In this study, volunteers received i.t. ketorolac, 0.25, 0.5, 1, or 2 mg, in an open label, dose-escalation design with 5 subjects per group. In early studies of 14 subjects, no side effects were observed. The purpose of this trial was safety assessment, but subjects were also screened for analgesic effects to acute noxious heat using a random staircase application of heat stimuli applied with a Peltier controlled thermode. There was an apparent effect with 0.5, but not 0.25 mg at both moderate and severe pain intensities (FIG. 3). I.t. ketorolac, 0.5 mg, did not affect temperature pain threshold in humans, consistent with lack of effect in withdrawal threshold to noxious heat in rats.

The two key observations of this study are that i.t. ketorolac, in doses up to 1.0 mg, produces no side effects and can cause several hours of analgesia to acute noxious heat. This safety trial did not provide for assessment of a time course of ketorolac's efficacy, but provided necessary data for power analysis for the proposed trial.

B. Efficacy in a Postoperative Model of Withdrawal Threshold

A model of tactile hypersensitivity after paw incision (Brennan, 1996) in male Sprague-Dawley rats was used to examine the effect of i.t. ketorolac, 50 μg, compared to saline. Pretreatment with i.t. ketorolac, 15 min before surgery increased withdrawal threshold compared to saline control 2 hr later and on the next day (FIG. 4). When given on the first postoperative day, at the time of established tactile hypersensitivity, i.t. ketorolac, but not saline, increased withdrawal threshold within 30 min (FIG. 4). Ketorolac's effect at this dose on withdrawal threshold was similar to that observed with IV morphine in this model (Zahn, 1997).

C. Efficacy in a Postoperative Model of Spontaneous and Motivated Behavior

A behavioral model was developed to assess pain following abdominal surgery. Briefly, rats are anesthetized and a 3 cm incision made in the right subcostal region, the small intestine manipulated, and the incision closed in three layers. Two types of experiments are performed: spontaneous (locomotor) activity and motivated (food-maintaining responding) activity. Locomotor activity is quantified in each animal using a computer-controlled system. Vertical counts are recorded by disruption of a bank of 24 infra-red beams located 7 inches above the floor surface. Ambulatory counts are recorded by disruption of two banks of 24 infra-red beams each in the X-Y plane 3 inches above the floor surface. For food-maintaining responding studies, lever presses are reinforced by presentation of standard 45 mg sucrose pellets. Animals are reduced to 85% of their free-feeding body weight and trained to press a lever for sucrose pellets using a fixed-ratio schedule during a 1 hr session using commercially-available operant equipment and customized software. The maximum number of pellets that can be obtained during each session is 200. The number of pellets earned and the time elapsed between the start of the session and the last pellet delivered are recorded.

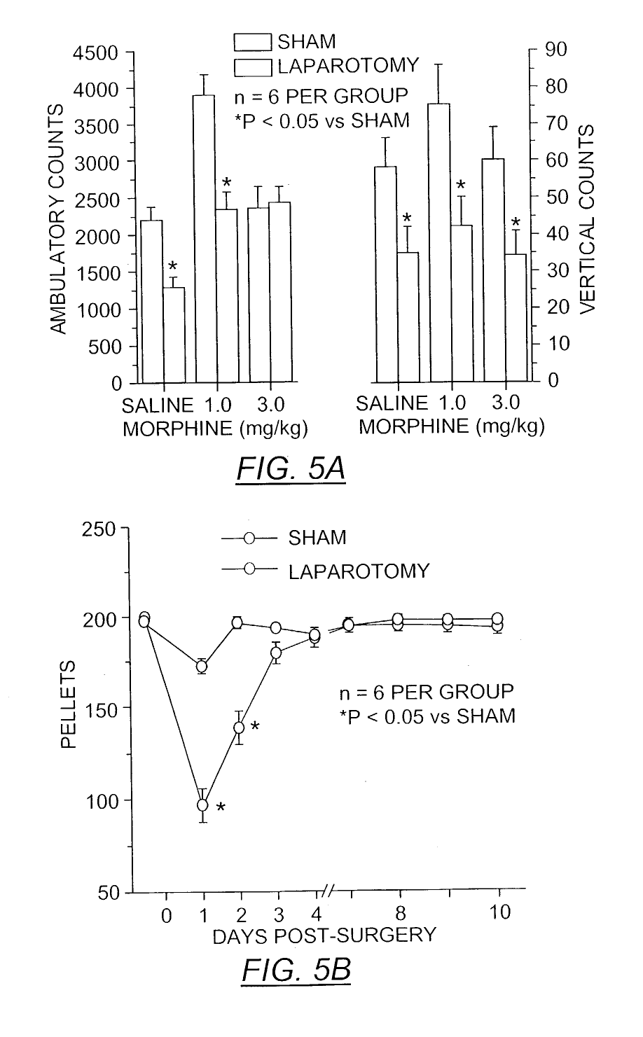

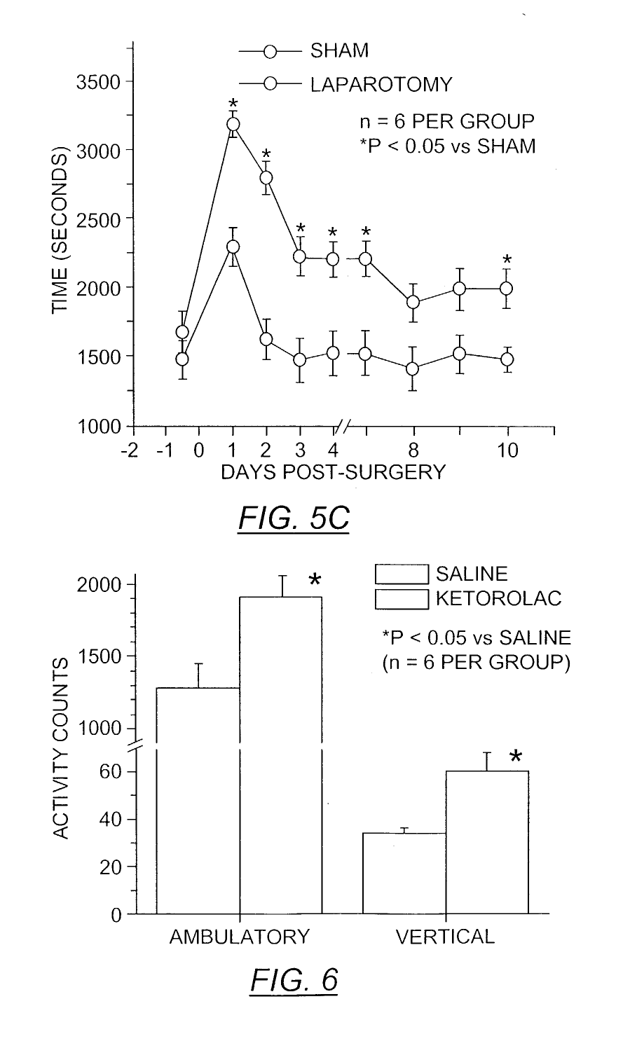

A series of validation studies were completed with this model. Briefly, laparotomy, but not sham (anesthetized and shaved, but no surgery) decreases locomotor activity one day after surgery, with gradual recovery to pre-surgery baseline over 3 days. The effect on ambulatory, but not vertical activity of laparotomy was reversed by IV morphine, 3 mg/kg (FIG. 5). Morphine at 1 mg/kg stimulates ambulatory activity in sham animals. Similarly, the number of sucrose pellets self-administered in a 1 hr trial session is reduced for 2 days following laparotomy, but not sham surgery (FIG. 5). The time required for animals to respond to earn food pellets is prolonged after laparotomy, but not sham surgery, and this effect is still present 10 days after surgery (FIG. 5).

In preliminary experiments with this model, i.t. ketorolac, 50 μg, increased both ambulatory and vertical locomotor activity after laparotomy compared to i.t. saline control (FIG. 6). The effect of ketorolac to increase ambulatory activity was similar to that observed with IV morphine, 3 mg/kg, and ketorolac increased vertical activity, which morphine did not (FIG. 5). Ketorolac alone in the absence of surgery had no effect on locomotor activity. In contrast, systemic ketorolac, 5 mg/kg i.p. failed to affect ambulatory activity, although it did potentiate morphine's effect, suggesting an increased efficacy as well as potency with i.t. administration. Ketorolac had a greater effect in this spontaneous activity model than in the Brennan model of withdrawal threshold, reinforcing initial clinical observations that i.t. ketorolac affects responses to a supra-threshold stimulus more than it does to alter threshold itself. The effect of i.t. ketorolac on food-maintenance responding behavior is being examined.

D. Effect of Surgery on Spinal COX Expression.

Spinal COX expression was evaluated in both Brennan and Martin models. In the Brennan model there is no change in COX-2 expression, as determined by immunocytochemistry of L5 spinal cord sections following surgery. In contrast, there is a large increase in COX-1 expression in cells with morphology consistent with microglia. This increase occurs throughout the dorsal horn laminae, not just in the superficial regions. This is consistent with studies using antibody coated microprobes which demonstrate that stimulated spinal PGE2 synthesis occurs uniformly from superficial to deep laminae (Ebersberger, 1999) and with previous studies demonstrating COX-1 is in spinal cord glial cells, but not neurons (Maihöfner, 2000). Consistent with the expression deep in the dorsal horn where Aβ fibers terminate, increased COX-1 immunostaining was also observed in the nucleus gracilis ipsilateral to surgery. Quantification was performed on these preliminary data.

In the Martin model there is an increase in COX-2 expression in the superficial dorsal horn, primarily in small neurons, but also in fibers. In addition, there is a large increase in COX-1 expression throughout the dorsal horn ipsilateral to the incision in structures resembling glia. It was confirmed that cfos immunostaining also increases in the spinal cord ipsilateral to surgical incision. Quantification in laminae I-III of the dorsal horn of low thoracic cord revealed a peak increase in COX-1 immunostaining ipsilateral to incision on postoperative day 1 (105±6 cells/section) compared to pre-surgery (46±5 cells), postoperative day 3 (73±11 cells/section) or day 7 (45±4 cells/section; P<0.05 on days 1 and 2 vs per-surgery). The number of COX-1 positive cells was only increased on postoperative day 1 compared to baseline (65±7 and 49±4 cells per section, respectively; P<0.05). A preliminary co-localization study in one animal with glial fibrillary acidic protein (GFAP) revealed no co-localization of this marker for astrocytes with COX-1, consistent with a morphology resembling microglia more than astrocytes.

### EXAMPLE 2

### Role of Spinal Cord COX-1 and COX-2 in Maintenance of Mechanical Hypersensitivity Following Peripheral Nerve Injury

A. Materials and Methods

Intrathecal Catheter Implantation

A Sprague-Dawley rats (Harlan Industries, Indianapolis, Ind., USA), weighing 200-250 g, were used in this study. All animal surgical procedures were in conformity with the Wake Forest University guidelines on the ethical use of animals and studies were approved by the Animal Care and Use Committee. Animals were implanted with intrathecal catheters according to the method described previously. (Yaksh TL, 1976). Briefly, under halothane anesthesia (2-4% in oxygen/air), animals were placed prone in a stereotaxic frame and a small incision was made at the back of the neck. A small puncture was made in the atlanto-occipital membrane of the cistema magna and a polyethylene catheter (PE-10, 7.5 cm) was inserted so that the caudal tip reached the lumbar enlargement of the spinal cord. The rostral end of the catheter was exteriorized at the top of the head and the wound was closed with sutures. Animals were allowed 4-5 days to recover from the surgery and those displaying signs of motor dysfunction (fore limb or hind limb paralysis) were excluded from the study.

Partial Sciatic Nerve Ligation and Behavioral Tests

Rats were anesthetized with 2-4% halothane in oxygen-air. For partial spinal nerve ligation (PSNL), the left sciatic nerve was exposed at the high thigh level and one-third to one-half of the nerve was ligated with silk suture (size 6) as previously described (Shir et al., 1991). Animals were maintained after surgery with ad libitum food and water on a 12-h light/dark cycle. All rats were allowed to recover for 4 weeks after PSNL. By then, tactile allodynia was well established in the ipsilateral hindpaw.

B. Results

Four weeks after PSNL, all rats developed allodynia in response to innocuous mechanical stimulation. Two hours following i.t. injection of ketorolac, the decreased withdrawal threshold was significantly reversed to pre-lesion baseline. The reversal lasted for 6 days. Eight days after i.t. injection, the withdrawal threshold value in the ipsilateral hindpaw returned to the pre-injection level, e.g., tactile allodynia reappeared.

In contrast to ketorolac, i.t. injection of piroxicam failed to attenuate well established tactile allodynia in either hindpaw. However, 2 and 4 h after i.t. injection of NS-398, the withdrawal threshold in the ipsilateral hindpaw was reversed to pre-lesion baseline level. The reversal lasted for 24 h and returned to pre-injection level at the time point of 48 h post-injection.

### EXAMPLE 3

### Effect of Local and Systemic Administration of Ketorolac of Tactile Allodynia Caused by Partial Sciatic Nerve Ligation

A. Materials and Methods

PSNL was performed as described above. Three weeks after PSNL, 0.2 mL 0.5% ketorolac (Syntex Inc., Palo Alto, Calif., USA) was injected (i) subcutaneously into the ipsilateral plantar side of the hindpaw, (ii) surrounding the ipsilateral injured nerve, (iii) into the ipsilateral biceps femoris muscle in the middle thigh, or (iv) intraperitoneally. Three rats in each group were also injected with normal saline to serve as controls. For all injections, rats were briefly anaesthetized by inhalation of 2-4% halothane/96% oxygen and air. Perineural injection was performed as previously described (Thalhammer et al. 1995). Briefly, the rat was held in lateral recumbency with the limb to be injected forming a right angle with a longitudinal axis of the trunk. The greater trochanter and ischial tuberosity were localized by palpation. On an imaginary line from the greater trochanter to the ischial tuberosity, about one third of the distance caudal to the greater trochanter, a 25-gauge injection needle was advanced from dorsolateral direction at a 45° angle until the tip encountered the ischium. A total volume of 0.2 mL was injected in a fanning motion along the path of the sciatic nerve. The withdrawal threshold to the stimulation of von Frey filaments was determined 3 h and 3 and 7 days after ketorolac injection.

B. Results

Three weeks after PSNL, 0.2 mL 0.5% ketorolac was intraplantarly injected into the plantar side of the ipsilateral footpad or into the injury site. Three hours to 5 days after PSNL, intraplantarly injected ketorolac reversed the tactile allodynia in the ipsilateral hindpaw of PSNL rats. Peri-neurally injected ketorolac had a slow onset of antiallodynic effect that was observed only 3 and 5 days after injection. Both intraplantar and peri-neural injection of saline had no effect on tactile allodynia.

Subsequently, studies were performed in order to determine whether systemic injection of ketorolac has an antiallodynic effect on well-developed tactile allodynia. Both intraperitoneal and intramuscular injections of 0.2 mL 0.5% ketorolac to rats 3 weeks after PSNL were performed. Three hours after intraperitoneal and intramuscular injection of ketorolac, the tactile allodynia in the ipsilateral paw was reversed. This reversal lasted for 3 days and disappeared by day 5 postinjection. Both intraperitoneal and intramuscular injection of saline had not effect on tactile allodynia.

It has been shown that the phosphorylation of cyclic AMP response element binding protein (CREB) was increased in the ipsilateral dorsal horn neurons 3 weeks after PSNL (Ma & Quirion, 2001). In this study, it was investigated whether local injection of ketorolac, which reversed tactile allodynia for more than 5 days, suppressed the increased number of phosphorylated CREB immunoreactive (pCREB-IR) cells in the dorsal horn of PSNL rats. Three weeks after PSNL and 5 days after intraplantar and peri-neural injection of ketorolac, the increased number of pCREB-IR cells in the ipsilateral dorsal horn of PSNL rats was dramatically reduced. The number of pCREB-IR cells in the contralateral dorsal horn after local injection of ketorolac was also decreased compared with that observed in saline injected PSNL rats. It appeared that the increased phosphorylation of CREB in the dorsal horn of PSNL rats was more dramatically suppressed by intraplantar than by peri-neural injection of ketorolac.

Quantitative analysis of the pCREB expression in the dorsal horn of PSNL rats receiving either saline or ketorolac intraplantar injection was conducted. As reported previously (Ma & Quirion, 2001), the mean number of pixels in a fixed area occupied by pCREB cells in the ipsilateral superficial dorsal horn of PSNL rats with saline injection was significantly increased compared to the contralateral side (P<0.001). However, no significant difference in the mean optical density in a fixed area occupied by pCREB-IR cells was detected between both sides of the dorsal horn. In PSNL rats with intraplantar ketorolac injection, the mean optical density in both the ipsilateral and contralateral dorsal horn was significantly decreased compared with that found in the respective counterparts obtained from saline injected rats (P<0.01).

### EXAMPLE 4

### Phase I Safety Assessment of Intrathecal Ketorolac

A. Materials and Methods

Participants

Twenty healthy volunteers were recruited by word of mouth and public, using wording approved by the Institutional Review Board (IRB). Only healthy adults (age 18-50), taking no medicines, specifically with no recent use of NSAIDs, without acute or chronic pain and not allergic to local anesthetics or ketorolac were included. The study was explained to them, all questions were answered, and written informed consent was obtained. The study and consent form were approved by the IRB, the FDA, and the Wake Forest University School of Medicine General Clinical Research Center (GCRC) Advisory Committee. Women were included in studies only after obtaining a negative pregnancy test and confirmation that they were not breast feeding.

Study Design: Safety

Volunteers arrived in the GCRC on the morning of study, having had nothing to eat or drink overnight. An 18 gauge cannula was inserted into a peripheral arm vein and lactated Ringers solution infused at 1.5 ml/kg/l for the duration of the study. Baseline measures included neurologic assessments (questioning for subjective sensations, screening examination of cranial nerve function, and testing upper and lower extremities for light touch and temperature sensation, motor strength, and deep tendon reflexes), blood pressure, heart rate, oxyhemoglobin saturation by pulse oximetry, and end-tidal CO₂. Neurologic assessments were repeated 45, 90, 150, 210, and 240 min after intrathecal injection, and the other measures were recorded at 15 and 30 min and 1, 2, 3, 4, and 24 hours (h) after intrathecal injection. Volunteers were actively questioned for side effects, specifically sedation, anxiety, gastrointestinal or genitourinary symptoms, dizziness, and weakness at the same time as the neurologic assessments, and also at 6 and 12 hours after intrathecal injection. Volunteers were contacted by telephone daily for 5 days, weekly for 1 month, and at 6 months after study and questioned regarding any side effects. The protocol included specific treatments to be used in the event of significant changes in blood pressure, heart rate, oxyhemoglobin saturation, respiration, or neurologic function.

Study Design: Efficacy

Efficacy was screened in this open-label trial using a Peltier-controlled thermode to apply heat stimuli. Volunteers were trained on a day prior to study, using a random staircase method, to consistently rate pain from a 5 second (s) increase in temperature from baseline (35° C.) to 39, 41, 43, 45, 46, 49, or 51° C. with probe temperatures separated by 25 seconds. Volunteers were not forced to use a 0-10 scale, but were instructed to apply numbers which were greater than zero only in the presence of pain and that reflected the degree of pain. This pain magnitude estimate has been previously validated (LaMotte et al., 1983).

Drug Administration and Cerebrospinal Fluid (CSF) Sampling

Preservative-free ketorolac (Acular P F, Allergan, Irvine, Calif., USA) was removed in a sterile fashion from its container and diluted to 2 ml with preservative-free saline. Lumbar puncture was performed following 1% lidocaine local infiltration using a 27. gauge Whitacre tipped needle at a lower lumbar interspace with the volunteer in the lateral decubitus position. Following collection of 5 ml CSF, the ketorolac solution was injected over 60 s, and the needle withdrawn. The volunteer was then positioned supine with the head of the bed elevated for comfort. Volunteers were allowed to ambulate after 1 h following injection, but remained in the GCRC for 4 h after injection and left in the care of a responsible adult. In this dose-escalation study, the first five volunteers received 0.25 mg, the next five received 0.5 mg, the next five received 1 mg, and the last five received 2 mg ketorolac. Escalation to the next dose was made only after review of side effects occurring at lower doses, with pre-defined stopping criteria.

CSF Assays

CSF samples were quantitatively extracted by C-18 reverse phase cartridge chromatography and eluted with acetonitrile. Concentrated eluates were injected on to a HPLC equipped with a Phenomenex ‘Prodigy’ C-18 reverse phase column (250 mm×4.6 mm). Peaks were detected with an Agilent Model 1100 UV detector set at a wavelength of 313. All unknowns, standards and controls contained an equal quantity of internal standard, 200 ng of indoprofen. With this assay, a single peak for ketorolac is found at the retention time of 10.68 min, the internal standard indoprofen elutes at 12.09 min. CSF extracts showed no interfering chromatography throughout the integration time period. The absolute sensitivity of the ketorolac assay was 5 ng/ml, and the coefficient of variation was <10% within the concentration range 5-500 ng/ml.

Data Analysis

Unless otherwise indicated, data are presented as mean±SE. continuous side effects data were analyzed by analysis of variance (ANOVA) for repeated measures followed by Dunnett's test to the control, pre-injection values. CSF concentrations were compared using a paired t-test. P<0.05 was considered significant.

B. Results

Safety

Intrathecal ketorolac had no effect on neurologic examination, and there were no subjective neurologic symptoms in any volunteer. All were able to ambulate normally when they were allowed to, and there was at no time a report of any subjective weakness. No volunteer reported sedation, anxiety, gastrointestinal or genitouriniary symptoms, or dizziness at any time when questioned or spontaneously. One individual, receiving the 0.5 mg dose of ketorolac had a mild headache 24 h after injection, which resolved the following day. No post-lumbar puncture headaches occurred. Long-teen follow up revealed no side effects.

Intrathecal ketorolac did not affect blood pressure, oxyhemoglobin saturation, or end-tidal CO₂, and all of these variables remained within 10% of pre-injection values. Heart rate decreased for 1 hr following ketorolac. This was significant, as determined by one-way ANOVA within each dose group except for the highest (2.0 mg) dose. In each case, post-hoc comparisons to baseline were not significant at any individual time within each dose group. When all subjects were taken together, the ANOVA was positive with significant reductions in heart rate at 15, 30, and 60 min after injection. As an entire group, heart rate prior to intrathecal ketorolac injection was 67±2.1 bpm (range 52-86 bpm), and the minimum heart rate at any time after injection was 57±1.7 bpm (range 45-69 bpm). The individual with the minimum heart rate of 45 bpm after ketorolac had a heart rate of 52 bpm before treatment. In no case did symptomatic bradycardia occur, and no volunteer met the criteria for treatment of bradycardia (heart rate <40 or <80% pre-injection, or with symptoms).

Efficacy

Threshold to heat pain in either the arm or the leg was unaffected by ketorolac as shown in Table 1 below. Similarly, there was no effect of any dose of ketorolac on response to suprathreshold stimuli. The pain report for the entire study population is depicted to probe temperatures from 43 to 51° C. for the arm and for the foot.

TABLE 1
Pain threshold temperatures in arm and leg before and after ketorolac^{a}

| Ketorolac | Time (h: injection at time 0) | | | | | | | |
|---|---|---|---|---|---|---|---|---|
| Dose | 0 | 0.25 | 0.5 | 1 | 2 | 3 | 4 | 24 |
| Arm | | | | | | | | |
| 0.25 mg | 45 ± 1.1 | 45 ± 1.0 | 44 ± 1.5 | 45 ± 1.3 | 44 ± 1.7 | 45 ± 1.1 | 44 ± 2.2 | 46 ± 0.5 |
| 0.50 mg | 45 ± 1.9 | 44 ± 1.9 | 43 ± 1.2 | 44 ± 1.7 | 45 ± 1.9 | 46 ± 1.7 | 46 ± 1.7 | 44 ± 1.7 |
| 1.0 mg | 40 ± 0.5 | 41 ± 1.3 | 40 ± 0.5 | 39 ± 0.7 | 41 ± 1.2 | 42 ± 0.9 | 42 ± 1.5 | 41 ± 1.6 |
| 2.0 mg | 44 ± 1.3 | 44 ± 1.9 | 43 ± 1.4 | 40 ± 2.1 | 43 ± 1.8 | 43 ± 1.6 | 43 ± 1.9 | 45 ± 2.3 |
| Leg | | | | | | | | |
| 0.25 mg | 45 ± 1.3 | 45 ± 1.1 | 46 ± 1.1 | 47 ± 0.7 | 46 ± 1.5 | 45 ± 1.1 | 46 ± 0.9 | 45 ± 1.1 |
| 0.50 mg | 45 ± 1.4 | 46 ± 2.4 | 48 ± 2.2 | 46 ± 1.7 | 46 ± 2.5 | 46 ± 1.7 | 46 ± 2.4 | 45 ± 2.0 |
| 1.0 mg | 41 ± 1.3 | 42 ± 1.7 | 42 ± 0.5 | 44 ± 0.5 | 42 ± 1.5 | 43 ± 1.0 | 43 ± 1.1 | 42 ± 0.9 |
| 2.0 mg | 45 ± 1.6 | 45 ± 1.4 | 43 ± 1.3 | 44 ± 2.1 | 44 ± 2.3 | 43 ± 1.6 | 44 ± 1.8 | 45 ± 2.2 |

^{a}Values are mean ± SE of five individuals. No difference in any group across time by repeated measures analysis of variance.

CSF Analyses

Ketorolac injection resulted 60 min later in detectable concentrations of drug in lumbar CSF, with an apparent plateau at the 1 and 2 mg doses as shown in Table 2 below. PGE2 concentrations in the entire study population averaged 4.9±0.5 pg/ml prior to ketorolac injection, and were not affected 60 min after ketorolac injection (6.1±0.6 pg/ml). In addition, there was no effect of any dose of ketorolac on CSF concentration of PGE2.

TABLE 2
Ketorolac and PGE2 concentrations in cerebrospinal fluid^{a}

| Ketorolac | CSF Ketorolac | Pre-injection | Post-injection |
|---|---|---|---|
| Dose | (μg/ml) | PGE2 (pg/ml) | PGE2 (pg/ml) |
| 0.25 mg | 4.6 ± 0.2 | 4.7 ± 0.7 | 6.0 ± 0.9 |
| 0.50 mg | 13.4 ± 1.2 | 5.6 ± 1.9 | 3.8 ± 0.6 |
| 1.0 mg | 30.7 ± 8.8 | 4.1 ± 0.9 | 8.5 ± 1.7 |
| 2.0 mg | 33.5 ± 5.2 | 4.9 ± 0.7 | 7.5 ± 1.7 |

^{a}Values are mean ± SE of five individuals. No difference in any group in PGE2 concentrations before and after injection by paired t-test.

### EXAMPLE 5

### Efficacy Assessment of Intrathecal Ketorolac

Intrathecal ketorolac was administered in an open label, dose escalation manner in patients with chronic pain receiving spinal morphine via an implanted, Medtronic pump and catheter system. This study focused on safety, but efficacy measures were obtained by having patients rate their pain using a visual analog scale (VAS) before, and at intervals after, intrathecal injection of ketorolac. Data are available from the first 6 patients in this open label study. The first 5 patients received 0.5 mg ketorolac, and the last patient received 1.0 mg ketorolac. There were no significant adverse events from ketorolac injection in this group. VAS pain was significantly reduced, as analyzed by one way ANOVA followed by Dunnett's test, beginning 1 hour after injection and lasting throughout the remaining 3 hours of observation. The reduction in VAS pain from 5 prior to injection, to 3 after injection is highly clinically significant in this patient group with chronic pain (*P<0.05 compared to time 0, n=6 (mean±SEM)). These data suggest that this agent is effective in humans with pain, just as it is in a variety of animal models of pain.

### EXAMPLE 6

### Intrathecal Ketorolac Enhances Antinociception from Clonidine

A. Materials and Methods

After Animal Care and Use Committee approval, adult male Sprague-Dawley rats (240-330 g; Harlan, Indianapolis, Ind.) were anesthetized with halothane. A catheter was inserted, as previously described, through a small nick in the cisterna magnum membrane into the intrathecal space and advanced 7.5 cm such that its tip lay in the lumbar intrathecal space. Rats recovered uneventfully, and proper catheter tip location was determined by appropriate bilateral lower-extremity motor block from an injection of 10 μl of 2% lidocaine through the catheter on the day after preparation. After catheter implantation, rats were housed individually with a 12 h/12 h light/dark cycle and an unlimited supply of water and food. Experiments occurred at least 6 days after surgery.

B. Results

All rats recovered uneventfully from surgery, and lidocaine produced bilateral motor block in all cases. There were no prolonged effects observed from any of the tested drugs or doses, nor was gross motor block observed from any of the test drug injections.

Clonidine produced antinociception, as measured by increased latency to paw withdrawal from noxious heat, whereas ketorolac did not. The addition of ketorolac to clonidine resulted in increased antinociception.

### EXAMPLE 7

### Intrathecal Ketorolac Reverses Hypersensitivity Following Acute Fentanyl Exposure

A. Materials and Methods

Animal Preparation and Fentanyl Administration

Male Harlan Sprague-Dawley rats weighing 225-275 g were used, and all procedures were approved by the Animal Care and Use Committee. For intrathecal drug administration, animals were anesthetized with halothane and a 32-gauge polyurethane catheter was inserted through a puncture of the atlanto-occipital membrane as previously described and advanced caudally so that the tip of the catheter was at the level of the lumbar enlargement. Animals that showed neurologic deficits were excluded from the study and euthanized immediately. After surgery, animals were housed individually and allowed to recover for 1 to 2 weeks.

Behavioral Tests

Three types of nociceptive tests were used, all measuring a withdrawal threshold. For thermal testing, a previously described method was used in which animals were acclimated at 30° C. A lamp was positioned under the hind paw, and when activated, focused light and radiant heat on the surface of the glass under the paw. Latency to withdrawal was determined before fentanyl exposure, and lamp intensity was adjusted to result in withdrawal with a latency of 10-15 s. Animals were tested 1, 2, and 4 days after fentanyl or saline exposure using the same lamp intensity as before drug injection. A cutoff of 30 s was not exceeded to avoid tissue injury. For mechanical testing, two methods were used. First, a commercially available device (Analgesymeter, Ugo Basile, Rome, Italy) was used to apply increasing pressure on a hind paw of the rat until paw withdrawal. A cutoff of 250 g was not exceeded to avoid tissue injury. Second, punctuate stimulation was used with von Frey filaments. For this, rats were placed in a Plexiglas box over a smooth mesh surface and allowed to acclimate for 30 min. A series of calibrated, hand made von Frey filaments (0.9-27.9 g), all with the, same diameter, were applied perpendicularly to the plantar surface of the left paw with a force to bend the filament for 5 s. Filaments of increasing force were applied until the rat withdraw its paw. Two minutes later, a filament of the next lesser force was applied, and threshold determined by the up-down method previously described. As with thermal tests, mechanical tests were performed before and 1, 2, and 4 days after subcutaneous injections. Six rats were tested with both thermal and von Frey methods, and six were tested for paw pressure.

Ketorolac Treatment

Preliminary experiments demonstrated that after fentanyl exposure, animals achieved cutoff levels of thermal mechanical stimulation for at least 3 h after injection, and had a maximal hypersensitivity to mechanical testing 1 day after fentanyl exposure. On the first day after fentanyl exposure, animals were randomized to receive intrathecal ketorolac, 5, 15, or 50 μg, with von Frey filament testing before and at 30 min intervals for 2 h after intrathecal injection (n=6 per group). The investigator was blinded to the ketorolac dose.

Immunocytochemistry

Rats were deeply anesthetized with pentobarbital and perfused pericardially with buffer (0.01 M phosphate buffered saline+1% sodium nitrite, 100 ml), followed by 4% paraformaldehyde (400 ml) either 24 or 96 h after fentanyl administration (n=4 at each time period). The L4-L6 portion of the spinal cord was extracted and submerged in 4% paraformaldehyde for 2 to 3 h followed by postfixation in 30% sucrose for 48-72 h at 4° C. Tissue was embedded in Tissue-Tek OCT Compound (Sakura Finetek, Torrance, Calif.) and cut transversely into 40 μm sections on a cryostat.

Immunocytochemistry was performed on free-floating sections using standard biotin-streptavidin techniques. After 4 washes with 0.01 M phosphate buffered saline+0.15% Triton 100× (PBS+T), sections were incubated in 0.3% hydrogen peroxide for 15 min. Sections were washed 4 times with PBS+T, incubated with 50% alcohol (45 min), washed 4 times with PBS+T and blocked with 1.5% normal serum. Sections were incubated in primary antibody, COX-1 monoclonal (1:1000; Cayman Chemicals, Ann Arbor, Mich.) or COX-2 polyclonal (1:5000; Cayman Chemicals), 24-48 h at 4° C. Sections were washed 4 times with PBS+T then incubated for 1 h with biotinylated secondary antibodies (1:200) and finally with horseradish peroxidase (HRP) conjugated tertiary antibody (1:100). Antibodies were visualized using the glucose-nickel-diaminobenizidine method images were captured on a light microscope at 10× magnification. Positively labeled cells were identified for automated counting using SigmaScan Pro 5 (Jandel Scientific, Carlsbad, Calif.) at a preset intensity threshold. Labeling was examined in a standardized area of the outer laminae (I-II) with 6-10 slices examined per animal.

Drugs

The following drugs were used: fentanyl citrate (Abbott Laboratories, Chicago, Ill.), and ketorolac tromethamine (Allergan, Irvine, Calif.). Ketorolac was diluted with normal saline and injected intrathecally in a volume of 10 μl over 30 s followed by 15 μl saline flush.

Statistics

Data are presented as mean±SE. Behavioral data were analyzed by either one-way or two-way repeated measures analysis of analysis of variance (ANOVA), followed by Dunnett test. Quantification of COX isoenzymes was compared by one-way ANOVA followed by Dunnett test. P<0.05 was considered significant.

B. Results

Behavioral Characterization of Fentanyl-Induced Hypersensitivity

Fentanyl, 320 μg/kg, first caused antinociception, then reduced withdrawal threshold to both measures of mechanical testing, but did not affect withdrawal threshold to heat. Hypersensitivity to mechanical testing was maximum on the first day after fentanyl exposure, and was still present to punctate, but not pressure testing 4 days after exposure. Hypersensitivity was greater to von Frey testing than to paw pressure testing, when expressed as percent reduction (57% vs. 26%), but not when expressed as reduction in multiples of the SD of the baseline (3.1-fold in both cases).

Effects of Intrathecal Ketorolac

Intrathecal ketorolac, 5 Hg, did not affect withdrawal threshold to von Frey filament testing, whereas 15, and 50 μg ketorolac increased withdrawal threshold for 30-60 min after injection. Two-way repeated measures ANOVA revealed a highly significant (P<0.001) dose-dependent effect from ketorolac, with each dose differing from the other. Animals appeared calm after intrathecal injections, with no alterations in spontaneous behavior.

Spinal Cox Isoenzyme Expression

COX-1 immunoreactivity (COX-1-IR) was localized exclusively within cells with glial morphology, and fentanyl administration did not alter this pattern of distribution. However, fentanyl administration significantly reduced the number of COX-1-IR cells at both 24 and 96 h (the number of labeled objects in laminae I and II per section was 73±1.4 in normal animals compared with 53±3.2 24 h after surgery, and 55±6.7 96 h after surgery; P<0.05 for both postsurgical times compared with normals).

COX-2 immunoreactivity was observed on the nuclei of neurons in the outer laminae with numerous perikarya being labeled throughout the dorsal horn. Motor neurons in the ventral horn were also immunoreactive. Fentanyl administration did not alter the immunoreactivity of COX-2 (number of COX-2 positive objects in laminae I and II in normals, animals at 24 h after surgery, and animals 96 h after surgery was 225±30; 208±42, and 263±55; P>0.05).

### EXAMPLE 8

### Intrathecal Lidocaine Reverses Tactile Allodynia Caused by Nerve Injuries and Potentiates the Antiallodynic Effect of Ketorolac

A. Materials and Methods

Intrathecal Catheter Implantation and Lidocaine Injection

A total of 26 male Sprague-Dawley rats (Harlan Industries, Indianapolis, Ind.), weighing 200-250 g, were used in this study. All surgical procedures were in conformity with the Wake Forest University (Winston-Salem, N.C.) guidelines on the ethical use of animals, and studies were approved by the Animal Care and Use Committee. Animals were implanted with intrathecal catheters according to the method described previously. Under halothane anesthesia (2-4% in oxygen-air), a polyethylene catheter (PE-10, 7.5 cm) was inserted intrathecally through a small puncture made in the atlanto-occipital membrane of the cisterna magna to reach the lumbar enlargement of the spinal cord. Animals were allowed 4 to 5 days to recover from the surgery, and those displaying signs of motor dysfunction (forelimb or hind limb paralysis) were excluded from the study. Lidocaine (100, 200, or 300 μg; Abbott Laboratories, Chicago, Ill.) was injected through the exteriorized portion of the catheter in 15 μl volume followed by a flush with 10 μl saline, 0.9%. Control rats were only injected with the same volume of saline. To determine whether systemically administered lidocaine is able to reverse established tactile allodynia, lidocaine (300 μg dissolved in 150 μl saline) was injected intraperitoneally in four PSNL rats 3 weeks following PSNL. To determine the effect of prior intrathecal lidocaine injection on the antiallodynic effect of the COX inhibitor, ketorolac, 1 week following intrathecal lidocaine injection (100-300 μg), 10 μl ketorolac (0.5%, 50 μg; Allergan, Irvine, Calif.) was intrathecally injected in these SNL rats.

Partial Sciatic Nerve Ligation, L5 and L6 Spinal Nerve Ligation, and Behavioral Tests

Rats were anesthetized with 2-4% halothane in oxygen-air. For PSNL, the left sciatic nerve was exposed at the high thigh level, and one third to one half of the nerve was ligated with 6-0 silk suture as previously described. For spinal nerve ligation (SNL), the left L5 and L6 spinal nerves were exposed and ligated with 6-0 silk suture as described before. Before and after surgery, all rats were behaviorally tested to determine the paw withdrawal threshold of both hind paws to mechanical stimuli. Animals were placed in a plastic cage with a wire mesh floor and allowed to explore and groom until they settled. A set of von Frey filaments with bending forces ranging from 1.25 to 30 g was applied, in ascending order, to both plantar hind paws (“up-and-down” method). A transient (10-20 min) weakness or paralysis of both hind limbs was seen in almost all rats with 300 μg intrathecal lidocaine injection but only in one third of rats with 200 μg intrathecal lidocaine and was completely absent in rats treated with 100 μg intrathecal lidocaine. Rats receiving either intrathecal saline or intraperitoneal lidocaine exhibited no abnormal behavior. Only after complete recovery from this paralysis were these rats tested behaviorally (2 h after intrathecal lidocaine). Each hind paw was measured three times, and the average values were obtained. Two independent individuals who were blinded to the study groups did the behavioral test. Similar results were obtained from the two examiners.

Statistical Analysis

The mean±SEM values from both hind paws were determined for each group. The mean values after nerve injury or after injection were compared with prelesion baseline values statistically using a one-way repeated measures analysis of variance with Dunnett multiple comparisons (SigmaStat, v. 2.03; Jandel Scientific Inc., San Rafael, Calif.). The significance level was set at P<0.05.

B. Results

Intrathecal Injection of Lidocaine Reverses Established Tactile Allodynia Caused by Partial Sciatic Nerve Ligation and Spinal Nerve Ligation

Two weeks after PSNL, the withdrawal threshold of both hind paws of all rats was significantly lower than the baseline value, indicating that tactile allodynia had developed. Then, 15 μl lidocaine (2%, 300 μg) was intrathecally injected in five rats, while saline in the same volume was injected in another five rats that served as controls. Another four rats received 300 μg intraperitoneal lidocaine. Two hours following intrathecal lidocaine, the tactile allodynia in both hind paws of intrathecal lidocaine-injected rats was significantly reversed. This reversal was also observed 3 days after intrathecal lidocaine. By then, intrathecal lidocaine also had an antinociceptive effect on the ipsilateral hind paw. However, 1 week after injection, the antiallodynic effect disappeared, and tactile allodynia was restored to preinjection level. At all time points following intrathecal saline injection, tactile allodynia was persistent in all rats. In four PSNL rats with intraperitoneal lidocaine, no attenuation or reversal of tactile allodynia was observed.

Four weeks following SNL, all rats exhibited a significant reduction in the withdrawal threshold of the ipsilateral hind paw when compared to the prelesion baseline. Two hours and 2 days following 100, 200, and 300 μg intrathecal lidocaine injection, tactile allodynia was markedly reversed to the prelesion level. Three days after injection, tactile allodynia reappeared in the ipsilateral hind paw of 200 and 300 μg intrathecal lidocaine-injected SNL rats. Although the withdrawal threshold in the hind paw of the 100 μg intrathecal lidocaine-injected SNL rats also declined, it was not significantly lower than the prelesion baseline value. The withdrawal threshold in the contralateral hind paw of all SNL rats was not significantly different from the prelesion level after either SNL or intrathecal lidocaine injection.

Intrathecal Lidocaine Potentiates the Antiallodynic Effect of Intrathecal Ketorolac in Spinal Nerve Ligation Rats

Consistent with a previous report, intrathecal ketorolac (50 μg) failed to attenuate the tactile allodynia caused by SNL. 1 week after intrathecal lidocaine, when tactile allodynia reappeared, intrathecal ketorolac reversed tactile allodynia for 4 h in SNL, rats that had previously received 200 and 300 μg lidocaine. One day after intrathecal ketorolac, its antiallodynic effect disappeared. However, intrathecal ketorolac failed to exert any antiallodynic effect on SNL rats that had received 100 μg intrathecal lidocaine previously. Intrathecal ketorolac (100 μg) alone also failed to alleviate SNL-induced tactile allodynia but also exhibited the antiallodynic effect 1 week after intrathecal lidocaine. The magnitude of the antiallodynic effect exerted by 100 μg intrathecal ketorolac was similar to that induced by 50 μg intrathecal ketorolac 1 week after prior intrathecal lidocaine injection. Either intrathecal saline injection 1 week following prior lidocaine injection or intrathecal ketorolac (50 μg) 1 week following prior intrathecal ketorolac (50 μg) injection failed to alleviate SNL-induced tactile allodynia.

The present invention can be embodied in different forms and should not be construed as limited to the embodiments set forth herein. Rather, these embodiments are provided so that this disclosure will be thorough and complete, and will fully convey the scope of the invention to those skilled in the art. All publications, patent applications, patents and other references cited herein are incorporated by reference in their entireties for the teachings relevant to the sentence and/or paragraph in which the reference is presented.

### REFERENCES

Baba H, Kohno T, Moore K A, and Woolf C J (2001) Direct activation of rat spinal dorsal horn neurons by prostaglandin E2. J. Neurosci. 21:1750-1756.

Ballou L R, Botting R M, Goorha S, Zhang J, and Vane J R (2000) Nociception in cyclooxygenase isoenzyme-deficient mice. Proc. Natl. Acad. Sci. USA 97:10272-10276.

Bennett D L, Michael G J, Ramachandran N, Munson J B, Averill S, Yan Q, McMahon S B, and Priestley J V (1998) A distinct subgroup of small DRG cells express GDNF receptor components and GDNF is protective for these neurons after nerve injury. J. Neurosci. 18:3059-3072.

Bickel A, Dorfs S, Schmelz M, Forster C, Uhl W, and Handwerker H O (1998) Effects of antihyperalgesic drugs on experimentally induced hyperalgesia in man. Pain 76:317-325.

Brennan T J, Umali E F, and Zahn P K (1997) Comparison of pre- versus post-incision administration of intrathecal bupivacaine and intrathecal morphine in a rat model of postoperative pain. Anesthesiology 87:1517-1528.

Brennan T J, Vandermeulen E P, and Gebhart G F (1996) Characterization of a rat model of incisional pain. Pain 64:493-501.

Bromage P R, Camporesi E, and Leslie J (1980) Epidural narcotics in volunteers: Sensitivity to pain and to carbon dioxide. Pain 9:145-160.

Bylund D B, Blaxall H S, Iversen L J, Caron M G, Letkowitz R J, and Lomasney J W (1992) Pharmacological characteristics of a₂-adrenergic receptors: Comparison of pharmacologically defined subtypes with subtypes identified by molecular cloning. Mol. Pharmacol. 42:1-5.

Célèrier E, Laulin J P, Corcuff J B, Le Moal M, and Simonnet G (2001) Progressive enhancement of delayed hyperalgesia induced by repeated heroin administration: A sensitization process. J. Neurosci. 21:4074-4080.

Célèrier E, Rivat C, Jun Y, Laulin J P, Larcher A, Reynier P, and Simonnet G (2000) Long-lasting hyperalgesia induced by fentanyl in rats—Preventive effect of ketamine. Anesthesiology 92:465-472.

Chaplan S R, Bach F W, Pogrel J W, Chung J M, and Yaksh T L (1994) Quantitative assessment of tactile allodynia in the rat paw. J. Neurosci. Methods 53:55-63.

Chandrasekharan N V, Dai H, Lamar K, Roos T, Evanson N K, Tomsik J, Elton T S, and Simmons D L (2002) Proc. Natl. Acad. Sci. USA, Vol. 99(21):13926-13931.

Chiari A, Li X H, Xu Z, Pan H L, and Eisenach J C (2000) Formation of 6-nitro-norepinephrine from nitric oxide and norepinephrine in the spinal cord and its role in spinal analgesia. Neuroscience 101:189-196.

Chiari A, Yaksh T L, Myers R R, Provencher J, Moore L, Lee C S, and Eisenach J C (1999) Preclinical toxicity screening of intrathecal adenosine in rats and dogs. Anesthesiology 91:824-832.

Coda B A, Brown M C, Schaffer R, Donaldson G, Jacobson R, Hautman B, and Shen D D (1994) Pharmacology of epidural fentanyl, alfentanil, and sufentanil in volunteers. Anesthesiology 81:1149-1161.

Colburn R W, DeLeo J A, Rickman A J, Yeager M P, Kwon P, and Hickey W F (1997) Dissociation of microglial activation and neuropathic pain behaviors following peripheral nerve injury in the rat. J. Neuroimmunol. 79:163-175.

Conklin D R and Eisenach J C (2003) Intrathecal ketorolac enhances antinociception from clonidine. Anesth. Analg. 96:191-194.

Detweiler D J, Eisenach J C, Tong C, and Jackson C (1993) A cholinergic interaction in alpha₂ adrenoceptor-mediated antinociception in sheep. J. Pharmacol. Exp. Ther. 265:536-542.

Dirig D M and Yaksh T L (1997) Hyperalgesia-associated spinal synthesis and release of prostaglandins. Adv. Exp. Med. Biol. 433:205-208.

Docquier, M.-A., De Kock, M., and Lavand'hommne, P. M. (2001) Spinal but not intravenous cyclo-oxygenase (COX) inhibitors reverse hyperalgesia consecutive to opioid administration in rats. Anesthesiology 95: A974

Dougherty P M and Willis W D (1992) Enhanced responses of spinothalamic tract neurons to excitatory amino acids accompany capsaicin-induced sensitization in the monkey. J. Neurosci. 12:883-894.

Dowlatshahi P and Yaksh T L (1997) Differential effects of two intraventricularly injected a₂ agonists, ST-91 and dexmedetomidine, on electroencephalogram, feeding, and electromyogram. Anesth. Analg. 84:133-138.

Ebersberger A, Grubb B D, Willingale H L, Gardiner N J, Nebe J, and Schaible H G (1999) The intraspinal release of prostaglandin E₂ in a model of acute arthritis is accompanied by an up-regulation of cyclo-oxygenase-2 in the spinal cord. Neuroscience 93:775-781.

Eisenach J C (2000a) Preemptive hyperalgesia, not analgesia? Anesthesiology 92:308-309.

Eisenach J C, D'Angelo R, Taylor C, and Hood D D (1994) An isobolographic study of epidural clonidine and fentanyl after cesarean section. Anesth. Analg. 79:285-290.

Eisenach J C, Curry R, Hood D D, and Yaksh T L (2002) Phase I safety assessment of intrathecal ketorolac. Pain 99:599-604.

Eisenach J C, DuPen S, Dubois M, Miguel R, Allin D, and Epidural Clonidine Study Group (1995a) Epidural clonidine analgesia for intractable cancer pain. Pain 61:391-399.

Eisenach J C and Gebhart G F (1995b) Intrathecal amitriptyline—Antiociceptive interactions with intravenous morphine and intrathecal clonidine, neostigmine, and carbamylcholine in rats. Anesthesiology 83:1036-1045.

Eisenach J C, Hood D D, and Curry R (1998) Intrathecal, but not intravenous, clonidine reduces experimental thermal or capsaicin-induced pain and hyperalgesia in normal volunteers. Anesth. Analg. 87:591-596.

Eisenach J C, Hood D D, and Curry R (2000b) Relative potency of epidural to intrathecal clonidine differs between acute thermal pain and capsaicin-induced allodynia. Pain 84:57-64.

Eisenach J C, Hood D D, Curry R, and Tong C Y (1997) Alfentanil, but not amitriptyline, reduces pain, hyperalgesia, and allodynia from intradermal injection of capsaicin in humans. Anesthesiology 86:1279-1287.

Eisenach J C and Tong C (1991) Site of hemodynamic effects of intrathecal a₂-adrenergic agonists. Anesthesiology 74:766-771.

Eissenberg T, Riggins E C, III, Harkins S W, and Weaver M F (2000) A clinical laboratory model for direct assessment of medication-induced antihyperalgesia and subjective effects: initial validation study. Exp. Clin. Psychopharmacol. 8:47-60.

Fu K Y, Light A R, and Maixner W (2000) Relationship between nociceptor activity, peripheral edema, spinal microglial activation and long-term hyperalgesia induced by formalin. Neuroscience 101:1127-1135.

Gomes I A, Li X H, Pan H L, and Eisenach J C (1999) Intrathecal adenosine interacts with a spinal noradrenergic system to produce antinociception in nerve-injured rats. Anesthesiology 91:1072-1079.

Goppelt-Struebe M and Beiche F (1997) Cyclooxygenase-2 in the spinal cord: Localization and regulation after a peripheral inflammatory stimulus. Adv. Exp. Med. Biol. 433:213-216.

Graham B A, Hammond D L, and Proudfit H K (2000) Synergistic interactions between two a₂-adrenoceptor agonists, dexmedetomidine and ST-91, in two substrains of Sprague-Dawley rats. Pain 85:135-143.

Guignard B, Bossard A E, Coste C, Sessler D I, Lebrault C, Alfonsi P, Fletcher D, and Chauvin M (2000) Acute opioid tolerance—Intraoperative remifentanil increases postoperative pain and morphine requirement. Anesthesiology 93:409-417.

Guo T Z, Davies M F, Kingery W S, Patterson A J, Limbird L E, and Maze M (1999) Nitrous oxide produces antinociceptive response via a_{2B} and/or a_{2C} adrenoceptor subtypes in mice. Anesthesiology; 90:470-476.

Gürün M S, Leinbach R, Moore L, Lee C S, Owen M D, and Eisenach J C (1997) Studies on the safety of glucose and paraben-containing neostigmine for intrathecal administration. Anesth. Analg. 85:317-323.

Hood D D, Mallak K A, James R L, Tuttle R, and Eisenach J C (1997) Enhancement of analgesia from systemic opioid in humans by spinal cholinesterase inhibition. J Pharmacol. Exp. Ther. 282:86-92.

Howe J R, Wang J Y, and Yaksh T L (1983) Selective antagonism of the antinociceptive effect of intrathecally applied alpha adrenergic agonists by intrathecal prazosiln and intrathecal yohimbine. J. Pharmacol. Exp. Ther. 224:552-558.

Huntoon M, Eisenach J C, and Boese P (1992) Epidural clonidine after cesarean section: Appropriate dose and effect of prior local anesthetic. Anesthesiology 76: 187-193.

Jasper J R, Lesnick J D, Chang L K, Yamanishi S S, Chang T K, Hsu S A, Daunt D A, Bonhaus D W, and Eglen R M (1998) Ligand efficacy and potency at recombinant a₂ adrenergic receptors—Agonist-mediated [³⁵S]GTPgammaS binding. Bio. Phar. 55:1035-1043.

Kang Y J, Vincler M, Li X, Conklin D, and Eisenach J C (2002) Intrathecal ketorolac reverses hypersensitivity following acute fentanyl exposure. Anesthesiology 97:1641-1644.

Karlsten R and Gordh T, Jr. (1995) An A1-selective adenosine agonist abolishes allodynia elicited by vibration and touch after intrathecal injection. Anesth. Analg. 80:844-847.

Kawahara H, Sakamoto A, Takeda S, Onodera H, Imaki J, and Ogawa R (2001) A prostaglandin e(2) receptor subtype ep(1) receptor antagonist (ono-8711) reduces hyperalgesia, allodynia, and c-fos gene expression in rats with chronic nerve constriction. Anesth. Analg. 93:1012-1017.

Kobinger W and Pichler L (1975) Investigation into some imidazoline compounds, with respect to peripheral a-adrenoceptor stimulation and depression of cardiovascular centers. Naunyn-Schmiedeberg's Arch. Pharmacol. 291:175-191.

Koppert, W., Alsheimer, M., Sittl, R., and Schmelz, M. Remifentanil-induced hyperalgesia in new human pain model. Anesthesiology 95, A861. 2001.

Koppert W, Likar R, Geisslinger G, Zeck S, Schmelz M, and Sittl R (1999) Peripheral antihyperalgesic effect of morphine to heat, but not mechanical, stimulation in healthy volunteers after ultraviolet-B irradiation. Anesth. Analg. 88:117-122.

LaBuda C J and Fuchs P N (2001) Low dose aspirin attenuates escape/avoidance behavior, but does not reduce mechanical hyperalgesia in a rodent model of inflammatory pain. Neurosci. Lett. 304:137-140.

LaMotte R H, Lundberg L E R, and Torebjork H E (1992) Pain, hyperalgesia and activity in nociceptive C units in humans after intradermal injection of capsaicin. J. Physiol. 448:749-764.

Lashbrook J M, Ossipova M H, Hunter J C, Raffa R B, Tallarida R J, and Porreca F (1999) Synergistic antiallodynic effects of spinal morphine with ketorolac and selective COX₁- and COX₂-inhibitors in nerve-injured rats. Pain 82:65-72.

Lauretti G R, Hood D D, Eisenach J C, and Pfeifer B L (1998) A multi-center study of intrathecal neostigmine for analgesia following vaginal hysterectomy. Anesthesiology 89:913-918.

Li X and Eisenach J C (2002) Nicotinic acetylcholine receptor regulation of spinal norepinephrine release. Anesthesiology. 96:1450-1456.

Li X and Eisenach J C (2001) a2A-Adrenoceptor stimulation reduces capsaicin-induced glutamate release from spinal cord synaptosomes. J. Pharmacol. Exp. Ther. 299:939-944.

Liu S S, Gerancher J C, Bainton B G, Kopacz D J, and Carpenter R L (1996) The effects of electrical stimulation at different frequencies on perception and pain in human volunteers: Epidural versus intravenous administration of fentanyl. Anesth. Analg. 82:98-102.

Lu J K, Schafer P G, Gardner T L, Pace N L, Zhang J, Niu S Y, Stanley T H, and Bailey P L (1997) The dose-response pharmacology of intrathecal sufentanil in female volunteers. Anesth. Analg. 85:372-379.

Ma W and Bisby M A (1998) Partial and complete sciatic nerve injuries induce similar increases of neuropeptide Y and vasoactive intestinal peptide immunoreactivities in primary sensory neurons and their central projections. Neuroscience 86:1217-1234.

Ma W and Quirion R (2001) Increased phosphorylation of cyclic AMP response element-binding protein (CREB) in the superficial dorsal horn neurons following partial sciatic nerve ligation. Pain 93:295-301.

Maihöfner C, Tegeder I, Euchenhofer C, Dewitt D, Brune K, Bang R, Neuhuber W, and Geisslinger G (2000) Localization and regulation of cyclo-oxygenase-1 and -2 and neuronal nitric oxide synthase in mouse spinal cord. Neuroscience 101: 1093-1108.

Malmberg A B and Yaksh T L (1992) Antinociceptive actions of spinal nonsteroidal anti-inflammatory agents on the formalin test in the rat. J. Pharmacol. Exp. Ther. 263:136-146.

Malmberg A B and Yaksh T L (1993) Pharmacology of the spinal action of ketorolac, morphine, ST-91, U50488H, and L-PIA on the formalin test and an isobolographic analysis of the NSAID interaction. Anesthesiology 79:270-281.

Malmberg A B and Yaksh T L (1994) Capsaicin-evoked prostaglandin E2 release in spinal cord slices: Relative effect of cyclooxygenase inhibitors. Eur. J. Pharmacol. 271:293-299.

McCormack K (1994) Non-steroidal anti-inflammatory drugs and spinal nociceptive processing. Pain 59:9-43.

McCormack K, Kidd B L, and Morris V (2000) Assay of topically administered ibuprofen using a model of post-injury hypersensitivity. Eur. J. Pharmacol. 56:459-462.

Meller S T, Dykstra C, and Gebhart G F (1996) Acute mechanical hyperalgesia in the rat can be produced by coactivation of spinal ionotropic AMPA and metabotropic glutamate receptors, activation of phospholipase A2 and generation of cyclooxygenase products. Prog. Brain Res. 110: 177-192.

Millan M J, Bervoets K, Rivet J-M, Widdowson P, Renouard A, Le Marotille-Girardon S, and Gobert A (1994) Multiple alpha-2 adrenergic receptor subtypes. II. Evidence for a role of rat alpha-2A adrenergic receptors in the control of nociception, motor behavior and hippocampal synthesis of noradrenaline. J. Pharmacol. Exp. Ther. 270:958-972.

Murakami M, Kambe T, Shimbara S, and Kudo I (1999) Functional coupling between various phospholipase A2s and cyclooxygenases in immediate and delayed prostanoid biosynthetic pathways. J. Biol. Chem. 274:3103-3115.

Nagasaka H and Yaksh TL (1990) Pharmacology of intrathecal adrenergic agonists: Cardiovascular and nociceptive reflexes in halothane-anesthetized rats. Anesthesiology 73:1198-1207.

Nakano H, Kobayashi T, Sugimoto Y, Ushikubi F, Ichikawa A, Narumiya S, and Ito S (2001) Characterization of EP receptor subtypes responsible for prostaglandin E2-induced pain responses by use of EP₁ and EP₃ receptor knockout mice. Br. J. Pharmacol. 133:438-444.

Okamoto K, Martin D P, Schmelzer J D, Mitsui Y, and Low P A (2001) Pro- and anti-inflammatory cytokine gene expression in rat sciatic nerve chronic constriction injury model of neuropathic pain. Exp. Neurol. 169:386-391.

Petersen K L, Jones B, Segredo V, Dahl J B, and Rowbotham M C (2001) Effect of remifentanil on pain and secondary hyperalgesia associated with the heat-capsaicin sensitization model in healthy volunteers. Anesthesiology 94:15-20.

Petersen K L and Rowbotham M C (1999) A new human experimental pain model: the heat/capsaicin sensitization model. Neuroreport 10: 1511-1516.

Price D D, Bush F M, Long S, and Harkins S W (1994) A comparison of pain measurement characteristics of mechanical visual analogue and simple numerical rating scales. Pain 56:217-226.

Price D D, Harkins SW, and Baker C (1987) Sensory-affective relationships among different types of clinical and experimental pain. Pain 28:297-307.

Price D D, McGrath P A, Rafil A, and Buckingham B (1983) The validation of visual analogue scales as ratio scale measures for chronic and experimental pain. Pain 17:45-56.

Price D D, McHaffie J G, and Larson M A (1989) Spatial summation of heat-induced pain: influence of stimulus area and spatial separation of stimuli on perceived pain sensation intensity and unpleasantness. J. Neurophysiol 62:1270-1279.

Puke M J C and Wiesenfeld-Hallin Z (1993) The differential effects of morphine and the a₂-adrenoceptor agonists clonidine and dexmedetomidine on the prevention and treatment of experimental neuropathic pain. Anesth. Analg. 77:104-109.

Ramwell P W, Shaw J E, and Jessup R (1966) Spontaneous and evoked release of prostaglandins from frog spinal cord. Am. J. Physiol. 211:998-1012.

Rice A S C, Lloyd J, Bullingham R E S, and O'Sullivan G (1993) Ketorolac penetration into the cerebrospinal fluid of humans. J. Clin. Anesth. 5:459-462.

Rosier, E. M., Iadarola, M. J., and Coghill, R. C. Reproducibility of pain measurement and pain perception. Am. Pain Soc. Abstracts 0, 140. 1999.

Sabbe M B, Grafe M R, Mjanger E, Tiseo P J, Hill H F, and Yaksh T L (1994) Spinal delivery of sufentanil, alfentanil, and morphine in dogs: Physiologic and toxicologic investigations. Anesthesiology 81:899-920.

Sabbe M B, Grafe M R, Pfeifer B L, Mirzai T H M, and Yaksh T L (1993) Toxicology of baclofen continuously infused into the spinal intrathecal space of the dog. NT 14:397-410.

Samad T A, Moore K A, Sapirstein A, Billet S, Allchorne A, Poole S, Bonventre J V, and Woolf C J (2001) Interleukin-1beta-mediated induction of Cox-2 in the CNS contributes to inflammatory pain hypersensitivity. Nature 410:471-475.

Sandner-Kiesling A, Pan H L, Chen S R, James R L, Dehaven-Hudkins D L, Dewan D M, and Eisenach J C (2001) Characterization of a new model of acute visceral nociception relevant to labor pain. Pain 96:13-22.

Shir Y and Seltzer Z (1991) Effects of sympathectomy in a model of causalgiform pain produced by partial sciatic nerve injury in rats. Pain 45:309-320.

Silverman D G, O'Connor T Z, and Brull S J (1993) Integrated assessment of pain scores and rescue morphine use during studies of analgesic efficacy. Anesth. Analg. 77:168-170.

Simone D A, Baumaun T K, and LaMotte R H (1989) Dose-dependent pain and mechanical hyperalgesia in humans after intradermal injection of capsaicin. Pain 38:99-107.

Sluka K A and Willis W D (1997) The effects of G-protein and protein kinase inhibitors on the behavioral responses of rats to intradermal injection of capsaicin. Pain 71:165-178.

Southall M D, Michael R L, and Vasko M R (1998) Intrathecal NSAIDS attenuate inflammation-induced neuropeptide release from rat spinal cord slices. Pain 178:39-48.

Stock J L, Shinjo K, Burkhardt J, Roach M, Taniguchi K, Ishikawa T, Kim H S, Flannery P J, Coffman T M, McNeish J D, and Audoly L P (2001) The prostaglandin E₂ EP1 receptor mediates pain perception and regulates blood pressure. J. Clin. Invest. 107:325-331.

Stone L S, Broberger C, Vulchanova L, Wilcox G L, Hökfelt T, Riedl M S, and Elde R (1998) Differential distribution of a_{2A} and a_{2C} adrenergic receptor immunoreactivity in the rat spinal cord. J. Neurosc. 18:5928-5937.

Stone L S, Vulchanova L, Riedl M S, Wang J, Williams F G, Wilcox G L, and Elde R (1999) Effects of peripheral nerve injury on alpha-2A and alpha-2C adrenergic receptor immunoreactivity in the rat spinal cord. Neuroscience 93:1399-1407.

Stubhaug A (1997) A new method to evaluate central sensitization to pain following surgery: Effect of ketamine. Acta Anaesth. Scand. Suppl. 110:154-155.

Sweitzer S M, Schubert P, and DeLeo J A (2001) Propentofylline, a glial modulating agent, exhibits antiallodynic properties in a rat model of neuropathic pain. J Pharmacol. Exp. Ther. 297:1210-1217.

Syriatowicz J-P, Hu D, Walker J S, and Tracey D J (1999) Hyperalgesia due to nerve injury: Role of prostaglandins. Neuroscience 94:587-594.

Taiwo Y O and Levine J D (1988) Prostaglandins inhibit endogenous pain control mechanisms by blocking transmission at spinal noradrenergic synapses. J. Neurosci. 8:1346-1349.

Takano Y and Yaksh T L (1992) Characterization of the pharmacology of intrathecally administered Alpha-2 agonists and antagonists in rats. J. Pharmacol. Exp. Ther. 261:764-772.

Takano Y and Yaksh T L (1993) Chronic spinal infusion of dexmedetomidine, ST-91 and clonidine: Spinal alpha₂ adrenoceptor subtypes and intrinsic activity. J. Pharmacol. Exp. Ther. 264:327-335.

Tallarida R J, Porreca F, and Cowan A (1989) Statistical analysis of drug-drug and site-site interactions with isobolograms. Life Sci. 45:947-961.

Thalhammer J G, Vladimirova M, Bershadsky B, and Strichartz G R (1995) Neurologic evaluation of the rat during sciatic nerve block with lidocaine. Anesthesiology 82: 1013-1025.

Tong C and Eisenach J C (2001) Preclinical examination of the safety of intrathecally administered ketorolac. Anesthesiology 95:A865.

Torebjork H E, Lundberg L E R, and LaMotte R H (1992) Central changes in processing of mechanoreceptive input in capsaicin-induced secondary hyperalgesia in humans. J. Physiol. 448:765-780.

Uda R, Horiguchi S, Ito S, Hyodo M, and Hayaishi O (1990) Nociceptive effects induced by intrathecal administration of prostaglandin D₂, E₂, or F₂ₐ to conscious mice. Brain Res. 510:26-32.

Ueda H, Matsunaga S, Inoue M, Yamamoto Y, and Hazato T (2000) Complete inhibition of purinoceptor agonist-induced nociception by spinorphin, but not by morphine. Peptides 21:1215-1221.

Versteeg H H, van Bergen en Henegouwen P M, van Deventer S J, and Peppelenbosch M P (1999) Cyclooxygenase-dependent signalling: molecular events and consequences. FEBS Lett. 445:1-5.

Vetter G, Geisslinger G, and Tegeder I (2001) Release of glutamate, nitric oxide and prostaglandin E₂ and metabolic activity in the spinal cord of rats following peripheral nociceptive stimulation. Pain 92:213-218.

Warner T D, Giuliano F, Vojnovic I, Bukasa A, Mitchell J A, and Vane J R (1999) Non-steroidal drug selectivities for cyclo-oxygenase-1 rather than cyclo-oxygenase-2 are associated with human gastrointestinal toxicity: a full in vitro analysis. Proc. Natl. Acad. Sci. USA 96:7563-7568.

Watkins L R, Martin D, Ulrich P, Tracey K J, and Maier S F (1997) Evidence for the involvement of spinal cord glia in subcutaneous formalin induced hyperalgesia in the rat. Pain 71:225-235.

Watkins L R, Milligan E D, and Maier S F (2001) Spinal cord glia: new players in pain. Pain 93:201-205.

Weiya M, Du W, and Eisenach J C (2003) Intrathecal lidocaine reverses tactile allodynia caused by nerve injuries and potentiates the antiallodynic effect of the COX inhibitor ketorolac. Anesthesiology 98:203-208.

Weiya M, Du W, and Eisenach J C (2002) Role for both spinal cord COX-1 and COX-2 in maintenance of mechanical hypersensitivity following peripheral nerve injury. Brain Research 937: 94-99.

Weiya M and Eisenach J C (2002) Morphological and pharmacological evidence for the role of peripheral prostaglandins in the pathogenesis of neuropathic pain. Eur. J. Neurosci. 15: 1037-1047.

Willingale H L, Gardiner N J, McLymont N, Giblett S, and Grubb B D (1997) Prostanoids synthesized by cyclo-oxygenase isoforms in rat spinal cord and their contribution to the development of neuronal hyperexcitability. Br. J. Pharmacol. 122:1593-1604.

Xu Z, Chen S R, Eisenach J C, and Pan H L (2000) Role of spinal muscarinic and nicotinic receptors in clonidine-induced nitric oxide release in a rat model of neuropathic pain. Brain Res. 861:390-398.

Yaksh T L, Dirig D M, Conway C M, Svensson C, Luo Z D, and Isakson P C (2001) The acute antihyperalgesic action of nonsteroidal, anti-inflammatory drugs and release of spinal prostaglandin E₂ is mediated by the inhibition of constitutive spinal cyclooxygenase-2 (COX-2) but not COX-1. J. Neurosci. 21:5847-5853.

Yaksh T L, Grafe M R, Malkmus S, Rathbun M L, and Eisenach J C (1995) Studies on the safety of chronically administered intrathecal neostigmine methylsulfate in rats and dogs. Anesthesiology 82:412-427.

Yaksh T L, Rathbun M, Jage J, Mirzai T, Grafe M, and Hiles R A (1994) Pharmacology and toxicology of chronically infused epidural clonidine.HCl in dogs. Fundam. Appl. Toxicol. 23:319-335.

Yaksh T L and Reddy S V R (1981) Studies in the primate on the analgesic effects associated with intrathecal actions of opiates, alpha adrenergic agonists and baclofen. Anesthesiology 54:451-467.

Yaksh T L and Rudy T A (1976) Chronic catheterization of the spinal subarachnoid space. Physiol. Behav. 7:1032-1036.

Yamamoto T and Sakashita Y (1999) The role of the spinal opioid receptor like1 receptor, the NK-1 receptor, and cyclooxygenase-2 in maintaining postoperative pain in the rat. Anesth. Analg. 89:1203-1208.

Yasuoka S and Yaksh T L (1983) Effects on nociceptive threshold and blood pressure of intrathecally administered morphine and a-adrenergic agonists. Neuropharmacol. 22:309-315.

Zahn P K, Gysbers D, and Brennan T J (1997) Effect of systemic and intrathecal morphine in a rat model of postoperative pain. Anesthesiology 86:1066-1077.

Zahn P K, Umali E, and Brennan T J (1998) Intrathecal non-NMDA excitatory amino acid receptor antagonists inhibit pain behaviors in a rat model of postoperative pain. Pain 74:213-223.

Zhang Z Z, Hefferan M P, and Loomis C W (2001) Topical bicuculline to the rat spinal cord induces highly localized allodynia that is mediated by spinal prostaglandins. Pain 92:351-361.

The foregoing is illustrative of the present invention, and is not to be construed as limiting thereof. The invention is defined by the following claims, with equivalents of the claims to be included therein.

What is claimed is:

1. A method of eliciting an analgesic effect in a subject in need thereof, comprising intrathecally administering to the subject a therapeutically effective amount of a cyclooxygenase 1 inhibitor or pharmaceutically acceptable salt thereof in a preservative-free pharmaceutically acceptable carrier.

2. The method of claim 1, wherein the cyclooxygenase 1 inhibitor is selected from the group consisting of ketorolac, piroxicam, and diclofenac and combinations thereof.

3. The method of claim 1, wherein the cyclooxygenase 1 inhibitor is administered with an adjuvant.

4. The method of claim 1, wherein the adjuvant is selected from the group consisting of an adrenergic agonist, opioid analgesic, local anesthetic, and calcium channel blocker, and combinations thereof.

5. The method of claim 3, wherein the adjuvant is clonidine, fentanyl, or lidocaine.

6. The method of claim 1, wherein the amount of the cyclooxygenase 1 inhibitor administered to the subject is from about 0.01 mg to about 5.0 mg.

7. A method of eliciting an analgesic effect in a subject in need thereof, comprising intrathecally administering a therapeutically effective amount of ketorolac or a pharmaceutically acceptable salt thereof to the subject in a preservative-free pharmaceutically acceptable carrier.

8. A pharmaceutical composition comprising:
a cyclooxygenase 1 inhibitor or a pharmaceutically acceptable salt thereof, and
an adjuvant selected from the group consisting of an adrenergic agonist, opioid analgesic, local anesthetic, and calcium channel blocker, and combinations thereof in a preservative-free pharmaceutically acceptable carrier.

9. The pharmaceutical composition of claim 8, wherein the cyclooxygenase 1 inhibitor is selected from the group consisting of ketorolac, piroxicam, and diclofenac and combinations thereof.

10. The pharmaceutical composition of claim 8, wherein the adjuvant is clonidine, fentanyl, or lidocaine.

11. A pharmaceutical composition comprising:
ketorolac or a pharmaceutically acceptable salt thereof, and
an adjuvant selected from the group consisting of an adrenergic agonist, opioid analgesic, local anesthetic, and calcium channel blocker, and combinations thereof in a preservative-free pharmaceutically acceptable carrier.

12. A pharmaceutical composition comprising ketorolac or a pharmaceutically acceptable salt thereof and clonidine in a preservative-free pharmaceutically acceptable carrier.

13. A pharmaceutical composition comprising ketorolac or a pharmaceutically acceptable salt thereof and fentanyl in a preservative-free pharmaceutically acceptable carrier.

14. A pharmaceutical composition comprising ketorolac or a pharmaceutically acceptable salt thereof and lidocaine in a preservative-free pharmaceutically acceptable carrier.

15. A method of eliciting an analgesic effect in a subject in need thereof, comprising intrathecally administering to the subject a therapeutically effective amount of a composition of claim 8.

16. A method of eliciting an analgesic effect in a subject in need thereof, comprising intrathecally administering to the subject a therapeutically effective amount of a composition of claim 9.

17. A method of eliciting an analgesic effect in a subject in need thereof, comprising intrathecally administering to the subject a therapeutically effective amount of a composition of claim 10

18. A method of eliciting an analgesic effect in a subject in need thereof, comprising intrathecally administering to the subject a therapeutically effective amount of a composition of claim 11.

19. A method of eliciting an analgesic effect in a subject in need thereof, comprising intrathecally administering to the subject a therapeutically effective amount of a composition of claim 12.

20. A method of eliciting an analgesic effect in a subject in need thereof, comprising intrathecally administering to the subject a therapeutically effective amount of a composition of claim 13.

21. A method of eliciting an analgesic effect in a subject in need thereof, comprising intrathecally administering to the subject a therapeutically effective amount of a composition of claim 14.

22. A kit comprising a composition comprising a cyclooxygenase 1 inhibitor or a pharmaceutically acceptable salt thereof in a preservative-free pharmaceutically acceptable carrier in a container suitable for delivery of the composition into an intrathecal administration device.

23. The kit of claim 22, wherein the cyclooxygenase 1 inhibitor is selected from the group consisting of ketorolac, piroxicam, and diclofenac, and combinations thereof.

24. The kit of claim 22, wherein the cyclooxygenase 1 inhibitor is ketorolac.

25. The kit of claim 22, further comprising an adjuvant.

26. The kit of claim 25, wherein the adjuvant is selected from the group consisting of an adrenergic agonist, opioid analgesic, local anesthetic, and calcium channel blocker, and combinations thereof in a preservative-free pharmaceutically acceptable carrier.

27. The kit of claim 22, wherein the adjuvant is clonidine, fentanyl, or lidocaine.

28. The kit of claim 22, wherein the intrathecal administration device is selected from the group consisting of a pump, syringe, catheter, and reservoir operably associated with a connecting device.

29. The kit of claim 28, wherein the intrathecal administration device is a pump.

30. A kit comprising a composition comprising ketorolac or a pharmaceutically acceptable salt thereof in a preservative-free pharmaceutically acceptable carrier in a container suitable for delivery of the composition into an intrathecal administration pump.
